(19) United States
(12) Patent Application Publication
(10) Pub. No.: US 2006/0270680 A1
(43) Pub. Date: Nov. 30, 2006

(54) Sulfonamide compounds and methods of making and using the same

(76) Inventors: Joel A Goldberg, Philadelphia, PA (US); Shili Chen, Chesire, CT (US); Jay J. Farmer, New Haven, CT (US); Alia Orbin, New Haven, CT (US); Joseph M. Salvino, Chester Spring, PA (US); Jiacheng Zhou, Hockessin, DE (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/559,222

(22) PCT Filed: Jun. 2, 2004
(86) PCT No.: PCT/US04/17097

§ 371 Date: Jun. 12, 2006

Related U.S. Application Data
(60) Provisional application No. 60/475,430, filed on Jun. 3, 2003.

(51) Int. Cl. A61K 31/506; A61K 31/501; A61K 31/497; A61K 31/4439; A61K 31/422; C07D 413/02

(52) U.S. Cl. 514/252.01; 514/252.05; 514/252.02; 514/255.05; 514/256; 514/340; 514/376; 514/378; 544/238; 544/333; 544/405; 546/271.4; 548/229

(57) ABSTRACT

The present invention relates generally to the field of anti-infective, anti-proliferative, anti-inflammatory, and prokinetic agents. More particularly, the invention relates to a family of compounds having both a biaryl moiety and at least one heterocylic moiety that are useful as such agents.

### RELATED APPLICATIONS

This application claims the benefit of and priority to U.S. Patent Application No. 60/475,430, filed Jun. 3, 2003, the enclosure of which is incorporated by reference herein.

### FIELD OF THE INVENTION

The present invention relates generally to the field of anti-infective, anti-proliferative, anti-inflammatory, and prokinetic agents. More particularly, the invention relates to a family of biaryl heterocyclic compounds, comprising both a biaryl moiety and at least one heterocyclic moiety, that are useful as therapeutic agents.

### BACKGROUND

Since the discovery of penicillin in the 1920s and streptomycin in the 1940s, many new compounds have been discovered or specifically designed for use as antibiotic agents. It was once believed that infectious diseases could be completely controlled or eradicated with the use of such therapeutic agents. However, such beliefs have been shaken by the fact that strains of cells or microorganisms resistant to currently effective therapeutic agents continue to evolve. In fact, virtually every antibiotic agent developed for clinical use has ultimately encountered problems with the emergence of resistant bacteria. For example, resistant strains of Gram-positive bacteria such as methicillin-resistant staphylocci, penicillin-resistant streptococci, and vancomycin-resistant enterococci have developed, which can cause serious and even fatal results for patients infected with such resistant bacteria Bacteria that are resistant to macrolide antibiotics, i.e., antibiotics based on a 14- to 16-membered lactone ring, have developed. Also, resistant strains of Gram-negative bacteria such as H. influenzae and M. catarrhalis have been identified. See, e.g., F. D. Lowry, “Antimicrobial Resistance: The Example of Staphylococcus aureus,” J. Clin. Invest., 2003, 111(9), 1265-1273; and Gold, H. S. and Moellering, R. C., Jr., “Antimicrobial-Drug Resistance,” N. Engl. J. Med, 1996, 335, 1445-53.

The problem of resistance is not limited to the area of anti-infective agents, because resistance has also been encountered with anti-proliferative agents used in cancer chemotherapy. Therefore, there exists a need for new anti-infective and anti-proliferative agents that are both effective against resistant bacteria and resistant strains of cancer cells.

In the antibiotic area, despite the problem of increasing antibiotic resistance, no new major classes of antibiotics have been developed for clinical use since the approval in the United States in 2000 of the oxazolidinone ring-containing antibiotic, N-[[(5S)-3-[3-fluoro-4-(4-morpholinyl)phenyl]-2-oxo-5-oxazolidinyl]methyl acetamide, which is known as linezolid and is sold under the tradename Zyvox® (see compound A). See, R. C. Moellering, Jr., “Linezolid: The First Oxazolidinone Antimicrobial,” Annals of Internal Medicine, 2003, 138(2), 135-142.

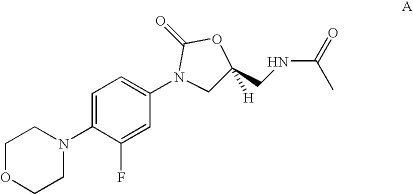

Linezolid was approved for use as an anti-bacterial agent active against Gram-positive organisms. Unfortunately, linezolid-resistant strains of organisms are already being reported. See, Tsiodras et al., Lancet, 2001, 358, 207; Gonzales et al., Lancet, 2001, 357, 1179; Zurenko et al., Proceedings Of The 39^{th} Annual Interscience Conference On Antibacterial Agents And Chemotherapy (ICAAC); San Francisco, Calif., USA, (Sep. 26-29, 1999). Because linezolid is both a clinically effective and commercially significant anti-microbial agent, investigators have been working to develop other effective linezolid derivatives.

Notwithstanding the foregoing, there is an ongoing need for new anti-infective and anti-proliferative agents. Furthermore, because many anti-infective and anti-proliferative agents have utility as anti-inflammatory agents and prokinetic agents, there is also an ongoing need for new compounds useful as anti-inflammatory and prokinetic agents.

### SUMMARY OF THE INVENTION

The invention provides a family of compounds useful as anti-infective agents and/or anti-proliferative agents, for example, chemotherapeutic agents, anti-microbial agents, anti-bacterial agents, anti-fungal agents, anti-parasitic agents, anti-viral agents, anti-inflammatory agents, and/or prokinetic (gastrointestinal modulatory) agents. The compounds have the formula: 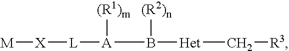
or a pharmaceutically acceptable salt, ester or prodrug thereof, wherein Het-CH₂—R³ is selected from the group consisting of: 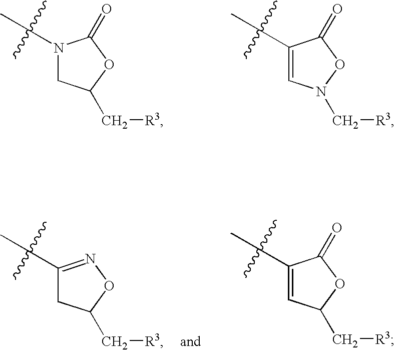
A and B independently are selected from the group consisting of phenyl, pyridyl, pyrazinyl, pyrimidinyl, and pyridazinyl; M is an optionally substituted C₁₋₆ alkyl, C₂₋₆ alkenyl, or C₂₋₆ alkynyl group; X is —SO₂NR⁴— or —NR⁴SO₂—; L is an optionally substituted C₁₋₆ alkyl, C₂₋₆ alkenyl, or C₂₋₆ alkynyl group; and the variables R¹, R², R³, R⁴, m, and n are selected from the respective groups of chemical moieties or integers later defined in the detailed description.

Particular embodiments of compounds of the invention include those having the formula: 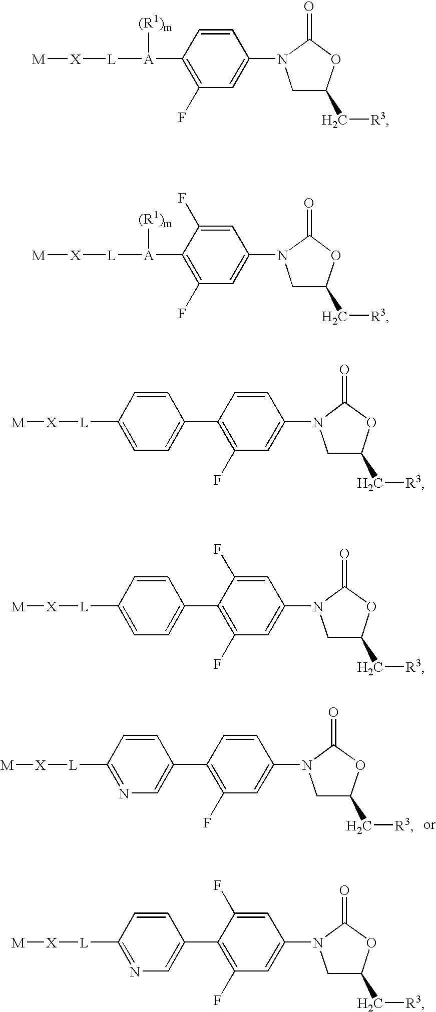
wherein the variables A, L, M, R¹, R³, X, and m are selected from the respective groups of chemical moieties or integers later defined in the detailed description.

In addition, the invention provides methods of synthesizing the foregoing compounds. Following synthesis, an effective amount of one or more of the compounds may be formulated with a pharmaceutically acceptable carrier for administration to a mammal for use as an anti-cancer, anti-microbial, anti-biotic, anti-fungal, anti-parasitic or anti-viral agent, or to treat a proliferative disease, an inflammatory disease or a gastrointestinal motility disorder. The compounds or formulations may be administered, for example, via oral, parenteral, or topical routes, to provide an effective amount of the compound to the mammal.

The foregoing and other aspects and embodiments of the invention may be more fully understood by reference to the following detailed description and claims.

### DETAILED DESCRIPTION OF THE INVENTION

The present invention provides a family of compounds that can be used as anti-proliferative agents and/or anti-infective agents. The compounds may be used without limitation, for example, as anti-cancer, anti-microbial, anti-bacterial, anti-fungal, anti-parasitic and/or anti-viral agents. Further, the present invention provides a family of compounds that can be used without limitation as anti-inflammatory agents, for example, for use in treating chronic inflammatory airway diseases, and/or as prokinetic agents, for example, for use in treating gastrointestinal motility disorders such as gastroesophageal reflux disease, gastroparesis (diabetic and post surgical), irritable bowel syndrome, and constipation.

### 1. DEFINITIONS

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

The present invention is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The compounds described herein may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, and geometric isomeric forms of a structure are intended, unless the specific stereochemistry or isomeric form is specifically indicated. All processes used to prepare compounds of the present invention and intermediates made therein are considered to be part of the present invention. All tautomers of shown or described compounds are also considered to be part of the present invention.

When any variable (e.g., R¹) occurs more than one time in any constituent or formula for a compound, its definition at each occurrence is independent of its definition at every other occurrence. Thus, for example, if a group is shown to be substituted with 0-2 R¹ moieties, then the group may optionally be substituted with up to two R¹ moieties and R¹ at each occurrence is selected independently from the definition of R¹. Also, combinations of substituents and/or variables are permissible, but only if such combinations result in stable compounds.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such substituent. Combinations of substituents and/or variables are permissible, but only if such combinations result in stable compounds.

Compounds of the present invention that contain nitrogens can be converted to N-oxides by treatment with an oxidizing agent (e.g., MCPBA and/or hydrogen peroxides) to afford other compounds of the present invention. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present invention can be converted to N-hydroxyl or N-alkoxyl compounds. For example, N-hydroxyl compounds can be prepared by oxidation of the parent amine by an oxidizing agent such as MCPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁₋₁₆ alkyl, alkenyl, alkynyl, C₃₋₁₄ carbocycle, or 3-14-membered heterocycle) derivatives.

When an atom or chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, s-pentyl, and n-hexyl.

As used herein, “alkenyl” is intended to include hydrocarbon chains of either straight or branched configuration having one or more carbon-carbon double bonds occurring at any stable point along the chain. For example, C₂₋₆ alkenyl is intended to include C₂, C₃, C₄, C₅, and C₆ alkenyl groups. Examples of alkenyl include, but are not limited to, ethenyl and propenyl.

As used herein, “alkynyl” is intended to include hydrocarbon chains of either straight or branched configuration having one or more carbon-carbon triple bonds occurring at any stable point along the chain. For example, C₂₋₆ alkynyl is intended to include C₂, C₃, C₄, C₅, and C₆ alkynyl groups. Examples of alkynyl include, but are not limited to, ethynyl and propynyl.

As used herein, “halo” or “halogen” refers to fluoro, chloro, bromo, and iodo. “Counterion” is used to represent a small, negatively charged species such as chloride, bromide, hydroxide, acetate, and sulfate.

As used herein, “carbocycle” or “carbocyclic ring” is intended to mean any stable monocyclic, bicyclic, or tricyclic ring having the specified number of carbons, any of which may be saturated, unsaturated, or aromatic. For example a C₃₋₁₄ carbocycle is intended to mean a mono-, bi-, or tricyclic ring having 3, 4, 5, 6, 7, 8, 9, 10, 11, 12, 13, or 14-carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, the term “heterocycle” or “heterocyclic” is intended to mean any stable monocyclic, bicyclic, or tricyclic ring which is saturated, unsaturated, or aromatic and comprises carbon atoms and one or more ring heteroatoms, e.g., 1 or 1-2 or 1-3 or 14 or 1-5 or 1-6 heteroatoms, independently selected from the group consisting of nitrogen, oxygen, and sulfur. A bicyclic or tricyclic heterocycle may have one or more heteroatoms located in one ring, or the heteroatoms may be located in more than one ring. The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “aromatic heterocycle” or “heteroaryl” is intended to mean a stable 5, 6, or 7-membered monocyclic or bicyclic aromatic heterocyclic ring or 7, 8, 9, 10, 11, or 12-membered bicyclic aromatic heterocyclic ring which consists of carbon atoms and one or more heteroatoms, e.g., 1 or 1-2 or 1-3 or 14 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluene sulfonic.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990).

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent.

As used herein, “treating” or “treatment” means the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

As used herein, “mammal” refers to human and non-human patients.

As used herein, the term “effective amount” refers to an amount of a compound, or a combination of compounds, of the present invention effective when administered alone or in combination as an anti-proliferative and/or anti-infective agent. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased anti-proliferative and/or anti-infective effect, or some other beneficial effect of the combination compared with the individual components.

All percentages and ratios used herein, unless otherwise indicated, are by weight.

Throughout the description, where compositions are described as having, including, or comprising specific components, it is contemplated that compositions also consist essentially of, or consist of, the recited components. Similarly, where processes are described as having, including, or comprising specific process steps, the processes also consist essentially of, or consist of, the recited processing steps. Further, it should be understood that the order of steps or order for performing certain actions are immaterial so long as the invention remains operable. Moreover, two or more steps or actions may be conducted simultaneously.

### 2. COMPOUNDS OF THE INVENTION

In one aspect, the invention provides compounds having the formula: 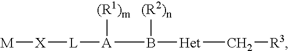
or a pharmaceutically acceptable salt, ester or prodrug thereof, wherein:

A is selected from the group consisting of:
phenyl, pyridyl, pyrazinyl, pyrimidinyl, and pyridazinyl;

B is selected from the group consisting of:
phenyl, pyridyl, pyrazinyl, pyrimidinyl, and pyridazinyl;

Het-CH₂—R³ is selected from the group consisting of:

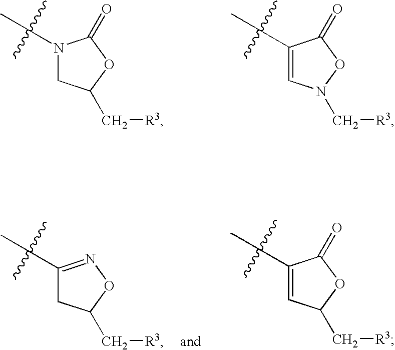

M is selected from the group consisting of:
a) C₁₋₄ alkyl, b) C₂₋₆ alkenyl, and c) C₂₋₆ alkynyl,
   wherein any of a)-c) optionally is substituted with one or more R⁴ groups;

X is selected from the group consisting of:
a) —SO₂NR⁴—, and b) —NR⁴SO₂—;

L is selected from the group consisting of:
a) C₁₋₆ alkyl, b) C₂₋₆ alkenyl, and c) C₂₋₆ alkynyl,
   wherein any of a)-c) optionally is substituted with one or more R⁴ groups;

R¹, at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR⁷C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —NR⁷C(NR⁷)NR⁷R⁷, aa) —S(O)ₚR⁷, bb) —SO₂NR⁷R⁷, and cc) R⁷;

R², at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR⁷C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —NR⁷C(NR⁷)NR⁷R⁷, aa) —S(O)ₚR⁷, bb) —SO₂NR⁷R⁷, and cc) R⁷;

R³ is selected from the group consisting of:
a) —OR⁷, b) —NR⁷R⁷, c) —C(O)R⁷, d) —C(O)OR⁷, e) —OC(O)R⁷, f) —C(O)NR⁷R⁷, g) —NR⁷C(O)R⁷, h) —OC(O)NR⁷R⁷, i) —NR⁷C(O)OR⁷, j) —NR⁷C(O)NR⁷R⁷, k) —C(S)R⁷, l) —C(S)OR⁷, m) —OC(S)R⁷, n) —C(S)NR⁷R⁷, o) —NR⁷C(S)R⁷, p) —OC(S)NR⁷R⁷, q) —NR⁷C(S)OR⁷, r) —NR⁷C(S)NR⁷R⁷, s) —NR⁷CR⁷)NR⁷R⁷, t) —S(O)ₚR⁷, u) —SO₂NR⁷R⁷, and v) R⁷;

R⁴, at each occurrence, independently is selected from the group consisting of:
a) H, b) F, c) Cl, d) Br, e) I, f) ═O, g) ═S, h) ═NR⁵, i) ═NOR⁵, j) ═N—NR⁵R⁵, k) —CF₃, l) —OR⁵, m) —CN, n) —NO₂, o) —NR⁵R⁵, p) —C(O)R⁵, q) —C(O)OR⁵, r) —OC(O)R⁵, s) —C(O)NR⁵R⁵, t) —NR⁵C(O)R⁵, u) —OC(O)NR⁵R⁵, v) —NR⁵C(O)OR⁵, w) —NR⁵C(O)NR⁵R⁵, x) —C(S)R⁵, y) —C(S)OR⁵, z) —OC(S)R⁵, aa) —C(S)NR⁵R⁵, bb) —NR⁵C(S)R⁵, cc) —OC(S)NR⁵R⁵, dd) —NR⁵C(S)OR⁵, ee) —NR⁵C(S)NR⁵R⁵, ff) —NR⁵C(NR⁵)NR⁵R⁵, gg) —S(O)ₚR⁵, hh) —SO₂NR⁵R⁵, and ii) R⁵;

R⁵, at each occurrence, independently is selected from the group consisting of:
a) H, b) C₁₋₆ alkyl, c) C₂₋₆ alkenyl, d) C₂₋₆ alkynyl, e) —C(O)—C₁₋₆ alkyl, f) —C(O)—C₂₋₆ alkenyl, g) —(O)—C₂₋₆ alkynyl, h) —C(O)O—C₁₋₆ alkyl, i) —C(O)O—C₂₋₆ alkenyl, and j) —C(O)O—C₂₋₆ alkynyl,
   wherein any of b)-j) optionally is substituted with one or more R⁶ groups;

R⁶, at each occurrence, independently is selected from the group consisting of:
a) F, b) Cl, c) Br, d) I, e) —CF₃, f) —OH, g) —OC₁₋₆ alkyl, h) —SH, i) —SC₁₋₆ alkyl, j) —CN, k) —NO₂, l) —NH₂, m) —NHC₁₋₆ alkyl, n) —N(C₁₋₆ alkyl)₂, o) —C(O)C₁₋₆ alkyl, p) —C(O)OC₁₋₆ alkyl, q) —C(O)NH₂, r) —C(O)NHC₁₋₆ alkyl, s) —C(O)N(C₁₋₆ allyl)₂, t) —NHC(O)C₁₋₆ alkyl, u) —SO₂NH₂, v) —SO₂NHC₁₋₆ alkyl, w) —SO₂N(C₁ alkyl)₂, and x) —S(O)ₚC₁₋₆ alkyl;

R⁷, at each occurrence, independently is selected from the group consisting of:
a) H, b) C₁₋₆ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur,
   wherein any of b)-p) optionally is substituted with one or more R⁸ groups;

R⁸, at each occurrence, is independently selected from the group consisting of:
a) F, b) Cl, c) Br, d) I, e) ═O, f) ═S, g) ═NR⁹, h) ═NOR⁹, i)═N—NR⁹R⁹, j) —CF₃, k) —OR⁹, l) —CN, m) —NO₂, n) —NR⁹R⁹, o) —C(O)R⁹, p) —C(O)OR⁹, q) —OC(O)R⁹, r) —C(O)NR⁹R⁹, s) —NR⁹C(O)R⁹, t) —OC(O)NR⁹R⁹, u) —NR⁹C(O)OR⁹, v) —NR⁹C(O)NR⁹R⁹, w) —C(S)R⁹, x) —C(S)OR⁹, y) —OC(S)R⁹, z) —C(S)NR⁹R⁹, aa) —NR⁹C(S)R⁹, bb) —OC(S)NR⁹R⁹, cc) —NR⁹C(S)OR⁹, dd) —NR⁹C(S)NR⁹R⁹, ee) —NR⁹C(NR⁹R⁹, ff) —S(O)ₚR⁹, gg)—SO₂NR⁹R⁹, and hh) R⁹;

R⁹, at each occurrence, independently is selected from the group consisting of:
a) H, b) C₁ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur,
   wherein any of b)-p) optionally is substituted with one or more moieties selected from the group consisting of:
      a) F, b) Cl, c) Br, d) I, e) —CF₃, f) —OH, g) —OC₁₋₆ alkyl, h) —SH, i) —SC₁₋₆ alkyl, j) —CN, k) —NO₂, l) —NH₂, m) —NHC-6 alkyl, n) —N(C₁₋₆ alkyl)₂, o) —C(O)C₁₋₆ alkyl, p) —C(O)OC₁₋₆ alkyl, q) —C(O)NH₂, r) —C(O)NHC₁₋₆ alkyl, s) —C(O)N(C₁₋₆ alkyl)₂, t) —NHC(O)C₁₋₆ alkyl, u) —SO₂NH₂, v) —SO₂NHC₁₋₆ alkyl, w) —SO₂N(C₁₋₆ alkyl)₂, and x) —S(O)ₚC₁₋₆ alkyl;

m is 0, 1, 2, 3, or 4;

n is 0, 1, 2, 3, or 4; and

p, at each occurrence, independently is 0, 1, or 2.

Particular embodiments of the invention include compounds having the formula: 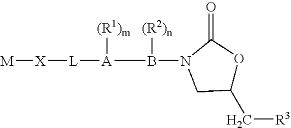
or a pharmaceutically acceptable salt, ester or prodrug thereof, wherein A, B, L, M, R¹, R², R³, X, m, and n are defined above.

Other embodiments include compounds having the formula: 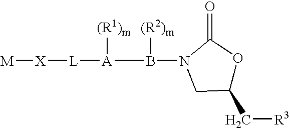
or a pharmaceutically acceptable salt, ester or prodrug thereof, wherein A, B, L, M, R¹, R², R³, X, m, and n are defined as described above.

Particular compounds include those where A is selected from the group consisting of phenyl and pyridyl; B is selected from the group consisting of phenyl and pyridyl; m is 0, 1, or 2; and n is 0, 1, or 2.

In some embodiments, A-B is: 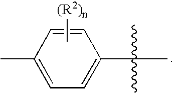
wherein A, R², and n are defined as described above. In particular embodiments, A-B is: 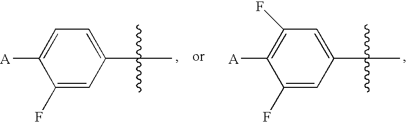
wherein A is defined as described above.

In various embodiments, A-B is: 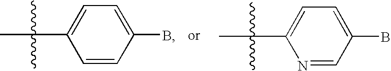
wherein B is defined as described in above.

In some embodiments, R³ is —NHC(O)R⁴. Particular compounds according to these embodiments include those where R⁴ is CH₃. In other embodiments, R³ is:

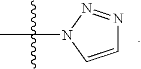

Particular embodiments of the invention include compounds having the formula: 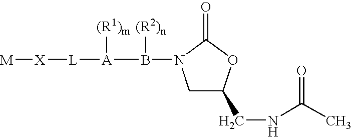
or a pharmaceutically acceptable salt, ester or prodrug thereof, wherein A, B, L, M, R¹, R², X, m, and n are defined as described above.

Other embodiments of the invention include compounds having the formula: 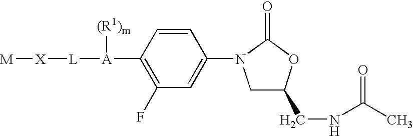
or a pharmaceutically acceptable salt, ester or prodrug thereof, wherein A, L, M, R¹, R³, X, and m are defined as described above.

Still other embodiments of the invention include compounds having the formula: 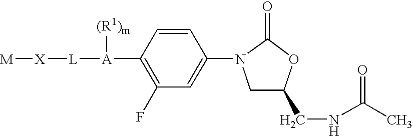
or a pharmaceutically acceptable salt, ester or prodrug thereof, wherein A, L, M, R¹, X, and m are defined as described above.

Some embodiments of the invention include compounds having the formula: 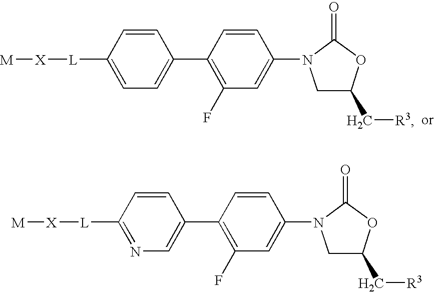
or a pharmaceutically acceptable salt, ester or prodrug thereof, wherein L, M, R³, and X are defined as described above.

Particular embodiments of the invention include compounds having the formula: 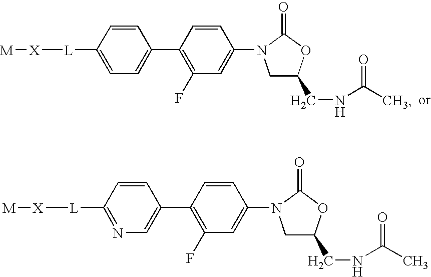
or a pharmaceutically acceptable salt, ester or prodrug thereof, wherein L, M, and X are defined as described above.

Other embodiments of the invention include compounds having the formula: 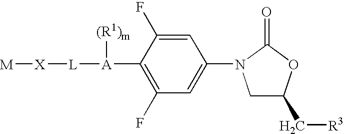
or a pharmaceutically acceptable salt, ester or prodrug thereof, wherein A, L, M, R¹, R³, X, and m are defined as described above.

Still other embodiments of the invention include compounds having the formula: 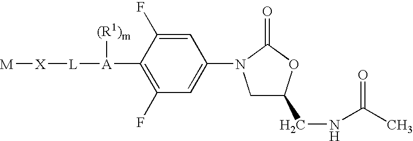
or a pharmaceutically acceptable salt, ester or prodrug thereof, wherein A, L, M, R¹, X, and m are defined as described above.

Some embodiments of the invention include compounds having the formula: 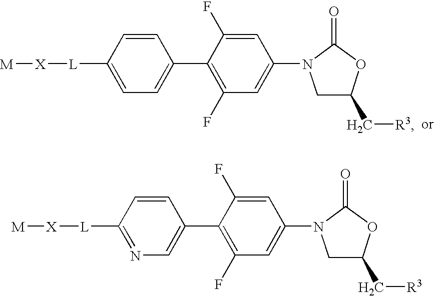
or a pharmaceutically acceptable salt, ester or prodrug thereof, wherein L, M, R³, and X are defined as described above.

Particular embodiments of the invention include compounds having the formula: 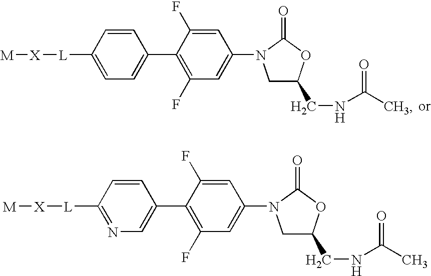
or a pharmaceutically acceptable salt, ester or prodrug thereof, wherein L, M, and X are defined as described above.

In some embodiments, L is C₁₋₆ alkyl. In particular embodiments, L is —CH₂—.

In other embodimetns, X is —SO₂NH—, —NHSO₂—, —SO₂NCH₃—, or —NCH₃SO₂—.

In certain embodiments, M is C₁₋₆ alkyl optionally substituted with one or more R⁴ groups. Particular embodiments include compounds wherein M is C₁₋₆ alkyl or C₁₋₆ alkyl substituted with one or more halogens.

In preferred embodiments, A is phenyl, substituted phenyl, pyridyl, or substituted pyridyl. In preferred embodiments, B is phenyl or substituted phenyl. More preferably, B is substituted phenyl. Preferred substituents include halogens, and in particular, fluorine.

In another aspect, the invention provides a pharmaceutical composition comprising an effective amount of one or more of the foregoing compounds and a pharmaceutically acceptable carrier. Suitable formulating agents are described in detail in section 5 hereinbelow.

One or more of the foregoing compounds may also be incorporated into a medical device. For example, a medical device, such as a medical stent, can contain or be coated with one or more of the compounds of the invention.

In another aspect, the invention provides a method for treating a microbial infection, a fungal infection, a viral infection, a parasitic disease, a proliferative disease, an inflammatory disease, or a gastrointestinal motility disorder in a mammal. The method involves administering an effective amount of one or more compounds or pharmaceutical compositions of the invention, for example, via oral, parenteral or topical routes.

The invention provides a method of treating a disorder in a mammal comprising the step of administering to the mammal an effective amount of one or more compounds of the invention thereby to ameliorate a symptom of a particular disorder. Such a disorder can be selected from the group consisting of a skin infection, nosocomial pneumonia, post-viral pneumonia, an abdominal infection, a urinary tract infection, bacteremia, septicemia, endocarditis, an atrio-ventricular shunt infection, a vascular access infection, meningitis, surgical prophylaxis, a peritoneal infection, a bone infection, a joint infection, a methicillin-resistant Staphylococcus aureus infection, a vancomycin-resistant Enterococci infection, a linezolid-resistant organism infection, and tuberculosis.

### 3. SYNTHESIS OF THE COMPOUNDS OF THE INVENTION

The invention provides methods and intermediates for making compounds of the present invention. The following schemes depict some exemplary chemistries available for synthesizing the compounds of the invention. It will be appreciated, however, that the desired compounds may be synthesized using other alternative chemistries known in the art.

The following examples illustrate certain compounds of the present invention. Compounds of general structures Ia and Ib (wherein X is CH or N) can be synthesized by the chemistries exemplified below in the following schemes.

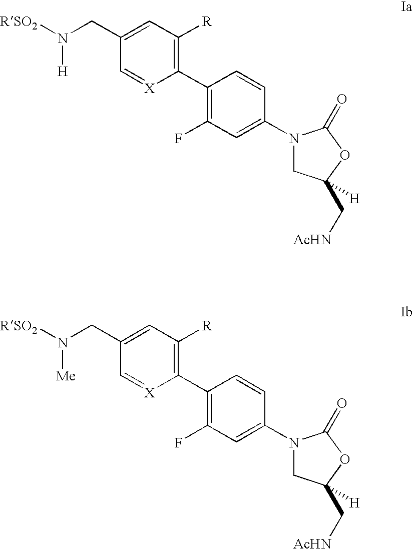

Scheme A exemplifies the synthesis of biaryl amine intermediate 5, which is useful in producing compounds of the present invention. Known iodoaryl oxazolidinone intermediate 1 (see U.S. Pat. Nos. 5,523,403 and 5,565,571) is coupled to a substituted aryl boronic acid (the Suzuki reaction) to produce biaryl alcohol 2. Other coupling reactions (for example, the Stille reaction), using alternate coupling intermediates easily obtained or synthesized by those skilled in the art, could also be employed to synthesize target biaryl intermediates similar to 2. These alternate coupling reactions are also within the scope of the present invention: Alcohol 2 is then converted to amine 5 by chemistry well known to those skilled in the art.

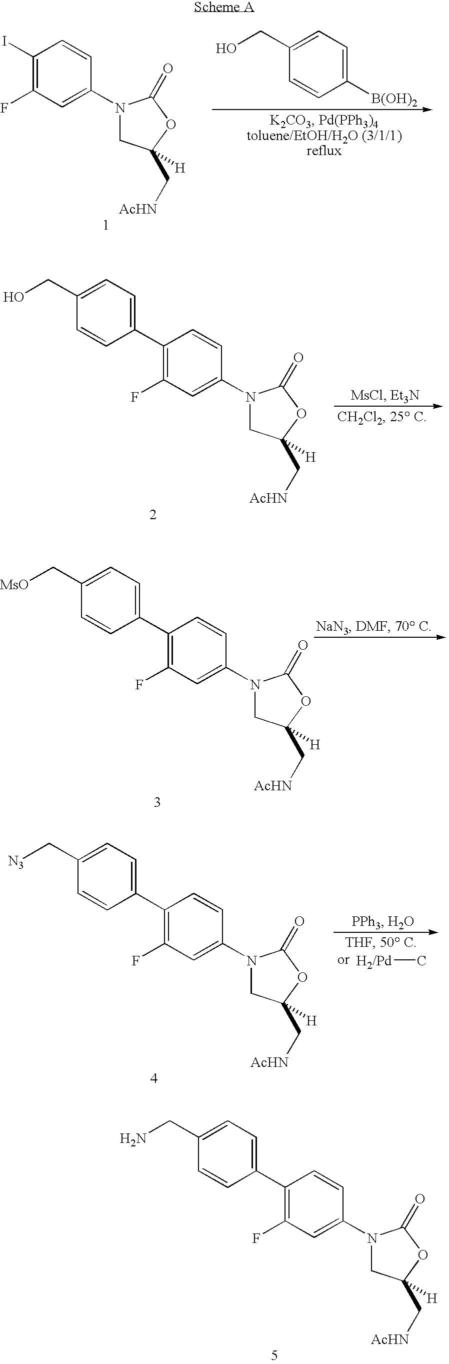

Scheme B illustrates the synthesis of intermediates 7 and 8 of the present invention, using Suzuki coupling chemistry between boronic acids and aryl triflates. Boronic ester 6 is treated with the appropriate aryl triflate to yield the BOC-protected biaryl 7. The BOC group of 7 is removed to provide amine 8, an intermediate useful in the synthesis of compounds of the present invention.

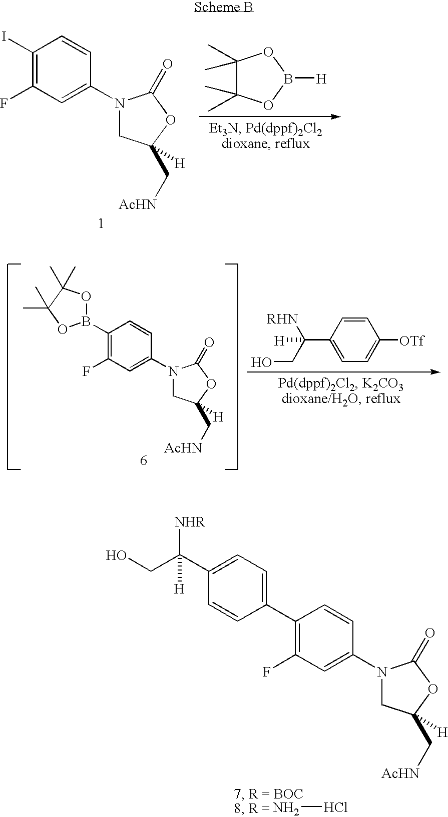

Scheme C depicts the synthesis of intermediates 9-13, which are useful in producing methoxy-substituted biaryl derivatives of the present invention. A Suzuki coupling of boronic ester 6 affords biaryl aldehyde 9, which can be reduced to alcohol 10. Mesylation of 10 yields 11, which can be converted to azide 12. Reduction of azide 12 yields amine 13.

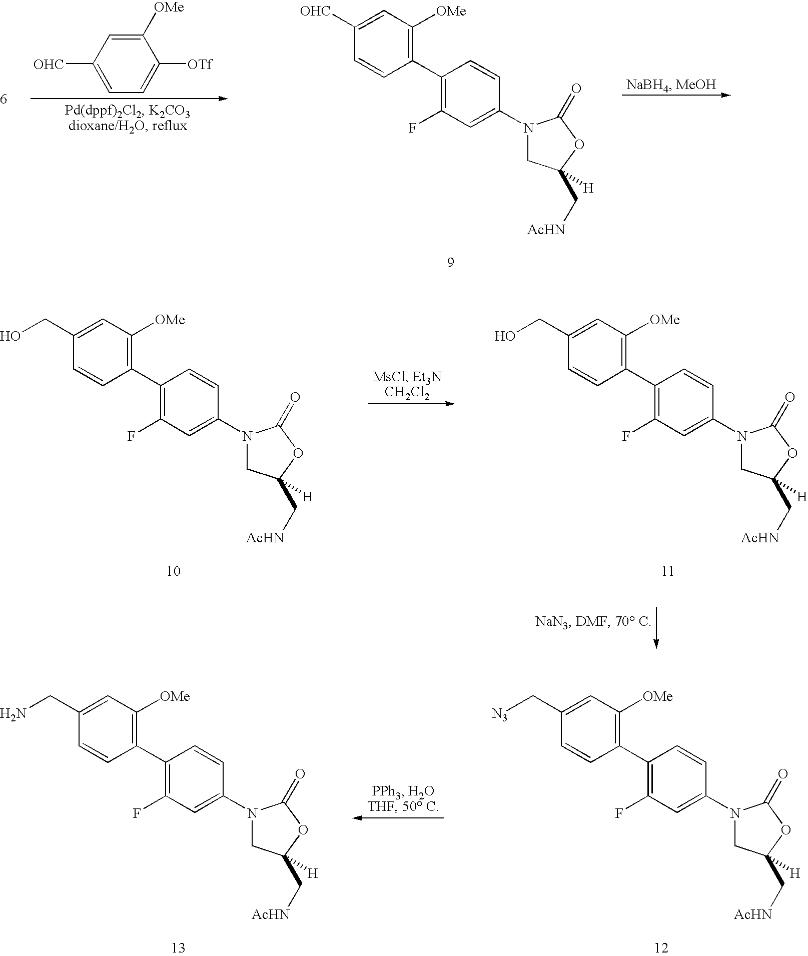

Scheme D depicts the synthesis of pyridyl intermediates, which are useful for the synthesis of compounds of the present invention, via similar chemistry to that shown in Scheme C. Coupling of boronic ester 6 to a halopyridine aldehyde affords biaryl aldehyde 14. Aldehyde 14 serves as the precursor to intermediates 15-18 via chemistry reported above.

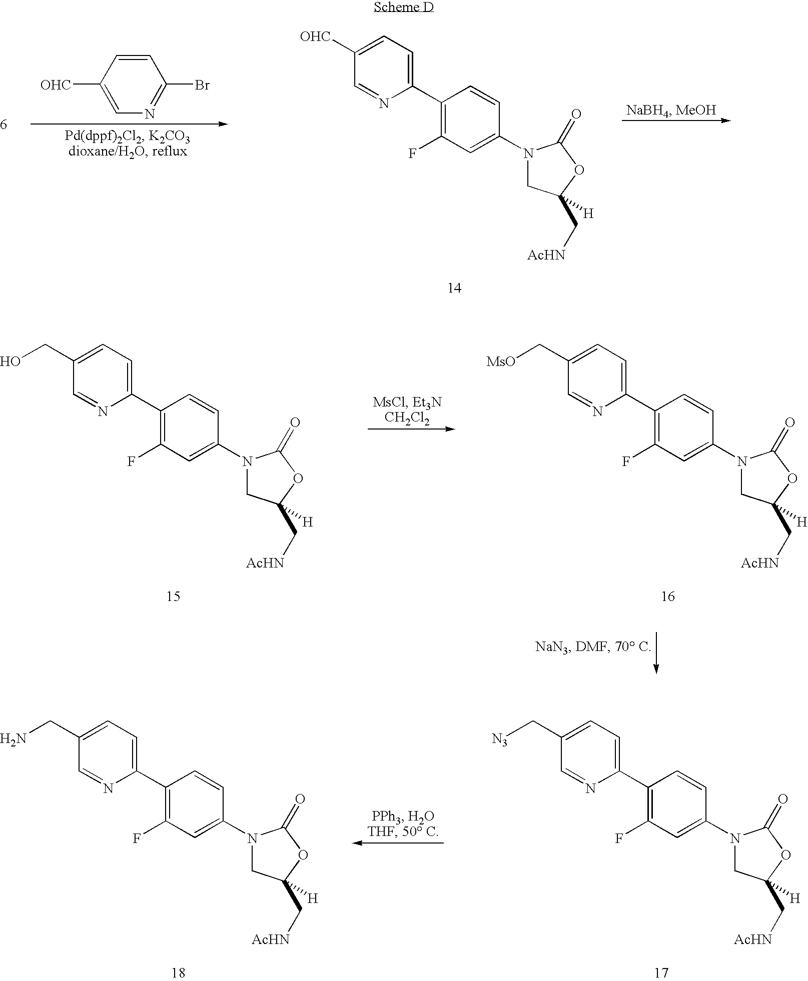

Biaryl aldehyde 19 (Scheme E) can be synthesized from a Suzuki coupling of iodide 1 and 4-formylphenylboronic acid. Scheme E illustrates how intermediate aldehydes of type 19, 9, and 14 can be converted via reductive amination chemistry to other amines such as 20-22, which are useful as intermediates for the synthesis of further compounds of the invention.

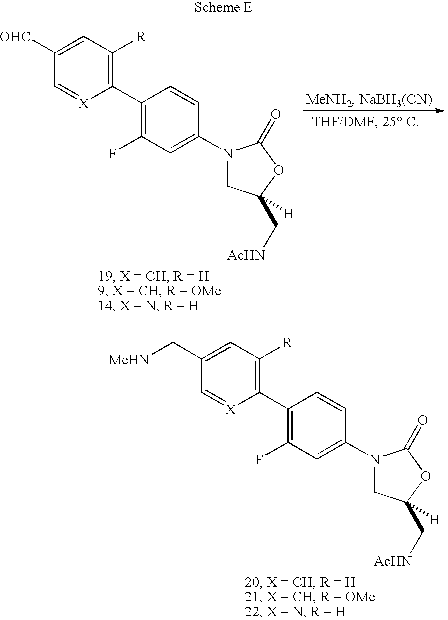

Scheme F illustrates the synthesis of sulfonamide derivatives of the present invention. Primary amines (such as 5, 13, and 18) and secondary amines (such as 20-21) can be directly converted to sulfonamides of general type Ia and Ib using a sulfonyl chloride in the presence of a suitable base. Alternatively, sulfonyl chlorides can be pre-loaded onto a solid polymeric support, such as a tetrafluorophenol containing resin (TFP resin) and reacted with amines to yield sulfonamide products of general structure Ia and Ib.

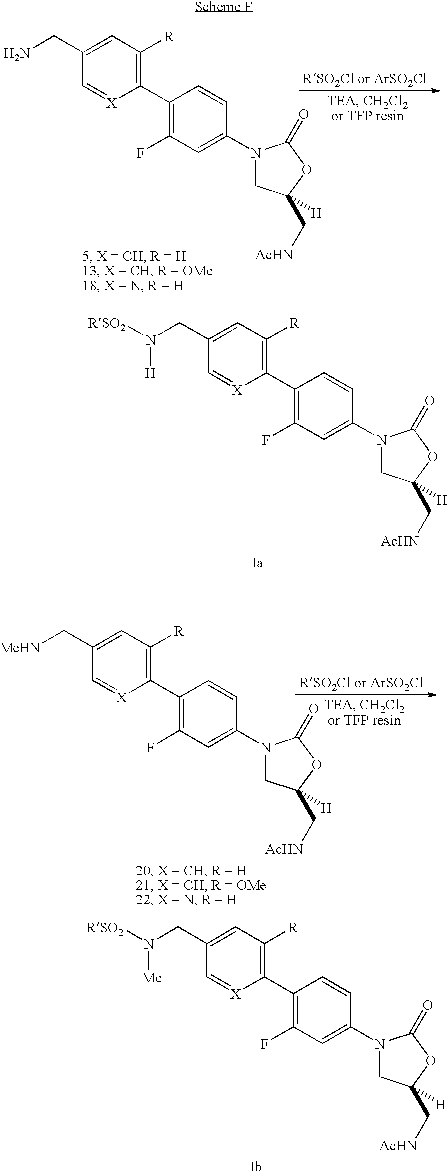

It should be noted that, when X is N, any of the synthetic routes described above may be used to produce compounds containing any regioisomer of pyridine (e.g., pyridin-2-yl or pyridin-3-yl).

### 4. CHARACTERIZATION OF COMPOUNDS OF THE INVENTION

Compounds designed, selected and/or optimized by methods described above, once produced, may be characterized using a variety of assays known to those skilled in the art to determine whether the compounds have biological activity. For example, the molecules may be characterized by conventional assays, including but not limited to those assays described below, to determine whether they have a predicted activity, binding activity and/or binding specificity.

Furthermore, high-throughput screening may be used to speed up analysis using such assays. As a result, it may be possible to rapidly screen the molecules described herein for activity, for example, as anti-cancer, anti-bacterial, anti-fungal, anti-parasitic or anti-viral agents. Also, it may be possible to assay how the compounds interact with a ribosome or ribosomal subunit and/or are effective as modulators (for example, inhibitors) of protein synthesis using techniques known in the art. General methodologies for performing high-throughput screening are described, for example, in Devlin, High Throughput Screening, Marcel Dekker (1998); and U.S. Pat. No. 5,763,263. High-throughput assays can use one or more different assay techniques including, but not limited to, those described below.

(1) Surface Binding Studies. A variety of binding assays may be useful in screening new molecules for their binding activity. One approach includes surface plasmon resonance (SPR) that can be used to evaluate the binding properties of molecules of interest with respect to a ribosome, ribosomal subunit or a fragment thereof.

SPR methodologies measure the interaction between two or more macromolecules in real-time through the generation of a quantum-mechanical surface plasmon. One device, (BIAcore Biosensor® from Pharmacia Biosensor, Piscatawy, N.J.) provides a focused beam of polychromatic light to the interface between a gold film (provided as a disposable biosensor “chip”) and a buffer compartment that can be regulated by the user. A 100 nm thick “hydrogel” composed of carboxylated dextran that provides a matrix for the covalent immobilization of analytes of interest is attached to the gold film. When the focused light interacts with the free electron cloud of the gold film, plasmon resonance is enhanced. The resulting reflected light is spectrally depleted in wavelengths that optimally evolved the resonance. By separating the reflected polychromatic light into its component wavelengths (by means of a prism), and determining the frequencies that are depleted, the BIAcore establishes an optical interface which accurately reports the behavior of the generated surface plasmon resonance. When designed as above, the plasmon resonance (and thus the depletion spectrum) is sensitive to mass in the evanescent field (which corresponds roughly to the thickness of the hydrogel). If one component of an interacting pair is immobilized to the hydrogel, and the interacting partner is provided through the buffer compartment, the interaction between the two components can be measured in real time based on the accumulation of mass in the evanescent field and its corresponding effects of the plasmon resonance as measured by the depletion spectrum. This system permits rapid and sensitive real-time measurement of the molecular interactions without the need to label either component.

(2) Fluorescence Polarization. Fluorescence polarization (FP) is a measurement technique that can readily be applied to protein-protein, protein-ligand, or RNA-ligand interactions in order to derive IC₅₀s and Kds of the association reaction between two molecules. In this technique one of the molecules of interest is conjugated with a fluorophore. This is generally the smaller molecule in the system (in this case, the compound of interest). The sample mixture, containing both the ligand-probe conjugate and the ribosome, ribosomal subunit or fragment thereof, is excited with vertically polarized light. Light is absorbed by the probe fluorophores, and re-emitted a short time later. The degree of polarization of the emitted light is measured. Polarization of the emitted light is dependent on several factors, but most importantly on viscosity of the solution and on the apparent molecular weight of the fluorophore. With proper controls, changes in the degree of polarization of the emitted light depends only on changes in the apparent molecular weight of the fluorophore, which in-turn depends on whether the probe-ligand conjugate is free in solution, or is bound to a receptor. Binding assays based on FP have a number of important advantages, including the measurement of IC₅₀s and Kds under true homogenous equilibrium conditions, speed of analysis and amenity to automation, and ability to screen in cloudy suspensions and colored solutions.

(3) Protein Synthesis. It is contemplated that, in addition to characterization by the foregoing biochemical assays, the compound of interest may also be characterized as a modulator (for example, an inhibitor of protein synthesis) of the functional activity of the ribosome or ribosomal subunit.

Furthermore, more specific protein synthesis inhibition assays may be performed by administering the compound to a whole organism, tissue, organ, organelle, cell, a cellular or subcellular extract, or a purified ribosome preparation and observing its pharmacological and inhibitory properties by determining, for example, its inhibition constant (IC₅₀) for inhibiting protein synthesis. Incorporation of ³H leucine or ³⁵S methionine, or similar experiments can be performed to investigate protein synthesis activity. A change in the amount or the rate of protein synthesis in the cell in the presence of a molecule of interest indicates that the molecule is a modulator of protein synthesis. A decrease in the rate or the amount of protein synthesis indicates that the molecule is a inhibitor of protein synthesis.

Furthermore, the compounds may be assayed for anti-proliferative or anti-infective properties on a cellular level. For example, where the target organism is a microorganism, the activity of compounds of interest may be assayed by growing the microorganisms of interest in media either containing or lacking the compound. Growth inhibition may be indicative that the molecule may be acting as a protein synthesis inhibitor. More specifically, the activity of the compounds of interest against bacterial pathogens may be demonstrated by the ability of the compound to inhibit growth of defined strains of human pathogens. For this purpose, a panel of bacterial strains can be assembled to include a variety of target pathogenic species, some containing resistance mechanisms that have been characterized. Use of such a panel of organisms permits the determination of structure-activity relationships not only in regards to potency and spectrum, but also with a view to obviating resistance mechanisms. The assays may be performed in microtiter trays according to conventional methodologies as published by The National Committee for Clinical Laboratory Standards (NCCLS) guidelines (NCCLS. M7-A5-Methods for Dilution Antimicrobial Susceptibility Tests for Bacteria That Grow Aerobically; Approved Standard-Fifth Edition. NCCLS Document M100-S12/M7 (ISBN 1-56238-394-9)).

### 5. FORMULATION AND ADMINISTRATION

The compounds of the invention may be useful in the prevention or treatment of a variety of human or other animal disorders, including for example, bacterial infection, fungal infections, viral infections, parasitic diseases, and cancer. It is contemplated that, once identified, the active molecules of the invention may be incorporated into any suitable carrier prior to use. The dose of active molecule, mode of administration and use of suitable carrier will depend upon the intended recipient and target organism. The formulations, both for veterinary and for human medical use, of compounds according to the present invention typically include such compounds in association with a pharmaceutically acceptable carrier.

The carrier(s) should be “acceptable” in the sense of being compatible with the other ingredients of the formulations and not deleterious to the recipient. Pharmaceutically acceptable carriers, in this regard, are intended to include any and all solvents, dispersion media, coatings, anti-bacterial and anti-fungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations may conveniently be presented in dosage unit form and may be prepared by any of the methods well known in the art of pharmacy/microbiology. In general, some formulations are prepared by bringing the compound into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, for example, intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, 18th ed. (Mack Publishing Company, 1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystalline form, for example, in the form of an aqueous microcrystalline suspension. Liposomal formulations or biodegradable polymer systems may also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling-powder-dispensing formulations. In the case of self-propelling solution and spray formulations, the effect may be achieved either by choice of a valve having the desired spray characteristics (i.e., being capable of producing a spray having the desired particle size) or by incorporating the active ingredient as a suspended powder in controlled particle size; For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in effective amounts, e.g., amounts that provide appropriate concentrations of the drug to target tissue for a time sufficient to induce the desired effect.

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

Active compound as identified or designed by the methods described herein can be administered to individuals to treat disorders (prophylactically or therapeutically). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a drug as well as tailoring the dosage and/or therapeutic regimen of treatment with the drug.

In therapeutic use for treating, or combating, bacterial infections in mammals, the compounds or pharmaceutical compositions thereof will be administered orally, parenterally and/or topically at a dosage to obtain and maintain a concentration, that is, an amount, or blood-level or tissue level of active component in the animal undergoing treatment which will be anti-microbially effective. The term “effective amount” is understood to mean that the compound of the invention is present in or on the recipient in an amount sufficient to elicit biological activity, for example, anti-microbial activity, anti-fungal activity, anti-viral activity, anti-parasitic activity, and/or anti-proliferative activity. Generally, an effective amount of dosage of active component will be in the range of from about 0.1 to about 100, more preferably from about 1.0 to about 50 mg/kg of body weight/day. The amount administered will also likely depend on such variables as the type and extent of disease or indication to be treated, the overall health status of the particular patient, the relative biological efficacy of the compound delivered, the formulation of the drug, the presence and types of excipients in the formulation, and the route of administration. Also, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

### 6. EXAMPLES

Exemplary compounds synthesized in accordance with the invention are listed in Table 1.

TABLE 1

| Compound | |
|---|---|
| Number | Structure |
| 101 | 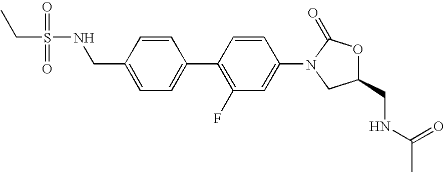 |
| | N-{3-[4′-(Ethanesulfonylamino-methyl)-2-fluoro-biphenyl-4- |
| | yl]-2-oxo-oxazolidin-5-(S)-ylmethyl}-acetamide |
| 102 | 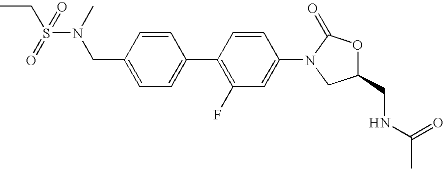 |
| | N-(3-{4′-[(Ethanesulfonyl-methyl-amino)-methyl]-2-fluoro- |
| | biphenyl-4-yl}-2-oxo-oxazolidin-5-(S)-ylmethyl)-acetamide |
| 103 | 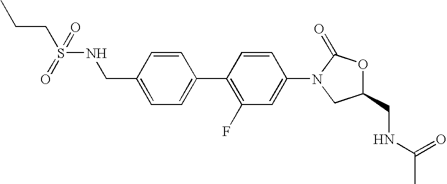 |
| | N-(3-{2-Fluoro-4′-[(propane-1-sulfonylamino)-methyl]- |
| | biphenyl-4-yl}-2-oxo-oxazolidin-5-(S)-ylmethyl)-acetamide |
| 104 | 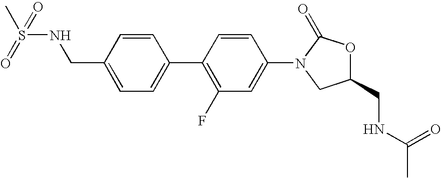 |
| | N-{3-[2-Fluoro-4′-(methanesulfonylamino-methyl)-biphenyl-4- |
| | yl]-2-oxo-oxazolidin-5-(S)-ylmethyl}-acetamide |
| 105 | 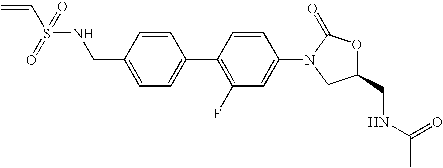 |
| | N-{3-[4′-(Ethenesulfonylamino-methyl)-2-fluoro-biphenyl-4- |
| | yl]-2-oxo-oxazolidin-5-(S)-ylmethyl}-acetamide |
| 106 | 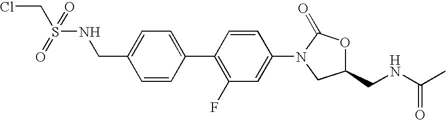 |
| | N-{3-[4′-(Chloromethanesulfonylamino-methyl)-2-fluoro- |
| | biphenyl-4-yl]-2-oxo-oxazolidin-5-(S)-ylmethyl}-acetamide |
| 107 | 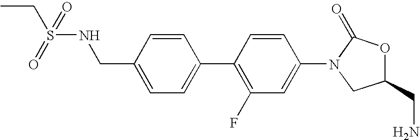 |
| | Ethanesulfonic acid [4′-(5-(S)-aminomethyl-2-oxo-oxazolidin- |
| | 3-yl)-2′-fluoro-biphenyl-4-ylmethyl]-amide |
| 108 | 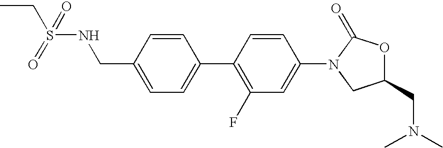 |
| | Ethanesulfonic acid [4′-(5-(S)-dimethylaminomethyl-2-oxo- |
| | oxazolidin-3-yl)-2′-fluoro-biphenyl-4-ylmethyl]-amide |
| 109 | 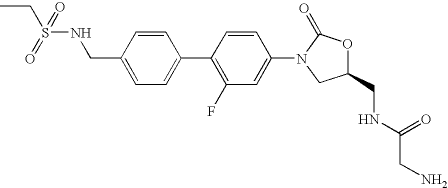 |
| | 2-Amino-N-{3-[4′-(ethanesulfonylamino-methyl)-2-fluoro- |
| | biphenyl-4-yl]-2-oxo-oxazolidin-5-(S)-ylmethyl}-acetamide |
| 110 | 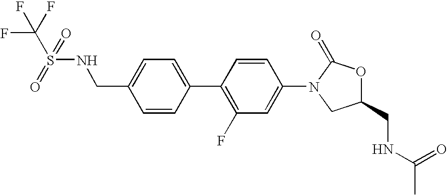 |
| | N-{3-[2-Fluoro-4′-(trifluoromethanesulfonylamino-methyl)- |
| | biphenyl-4-yl]-2-oxo-oxazolidin-5-(S)-ylmethyl}-acetamide |
| 111 | 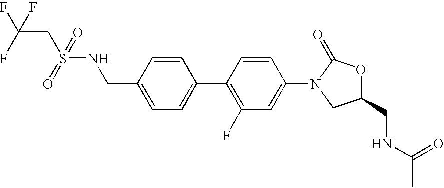 |
| | N-(3- {2-Fluoro-4′-[(2,2,2-trifluoro-ethanesulfonylamino)- |
| | methyl]-biphenyl-4-yl}-2-oxo-oxazolidin-5-(S)-ylmethyl)- |
| | acetamide |
| 112 | 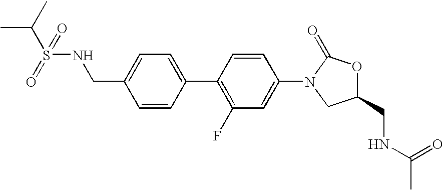 |
| | N-(3-{2-Fluoro-4′-[(propane-2-sulfonylamino)-methyl]- |
| | biphenyl-4-yl}-2-oxo-oxazolidin-5-(S)-ylmethyl)-acetamide |
| 113 | 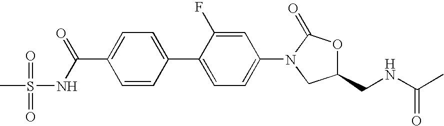 |
| | N-[3-(2-Fluoro-4′-methanesulfonylaminocarbonyl-biphenyl-4- |
| | yl)-2-oxo-oxazolidin-5-(S)-ylmethyl]-acetamide |
| 114 | 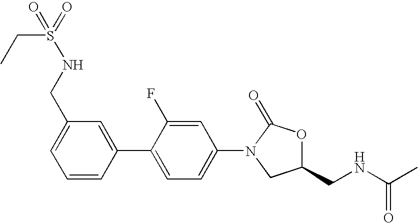 |
| | N-{3-[3′-(Ethanesulfonylamino-methyl)-2-fluoro-biphenyl-4- |
| | yl]-2-oxo-oxazolidin-5-(S)-ylmethyl}-acetamide |
| 115 | 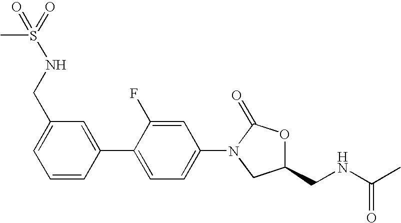 |
| | N-{3-[2-Fluoro-3′-(methanesulfonylamino-methyl)-biphenyl-4- |
| | yl]-2-oxo-oxazolidin-5-(S)-ylmethyl}-acetamide |
| 116 | 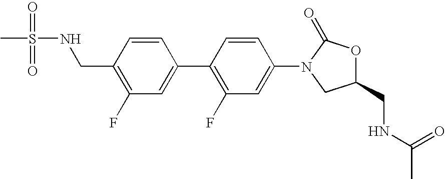 |
| | N-{3-[2,3′-Difluoro-4′-(methanesulfonylamino-methyl)- |
| | biphenyl-4-yl]-2-oxo-oxazolidin-5-(S)-ylmethyl}-acetamide |
| 117 | 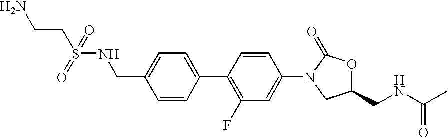 |
| | N-(3-{4′-[(2-Amino-ethanesulfonylamino)-methyl]-2-fluoro- |
| | biphenyl-4-yl}-2-oxo-oxazolidin-5-(S)-ylmethyl)-acetamide |
| 118 | 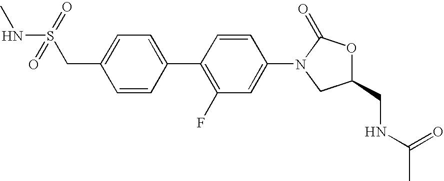 |
| | N-[3-(2-Fluoro-4′-methylsulfamoylmethyl-biphenyl-4-yl)-2- |
| | oxo-oxazolidin-5-(S)-ylmethyl]-acetamide |

Nuclear magnetic resonance (NMR) spectra were obtained on a Bruker Avance 300 or Avance 500 spectrometer, or in some cases a GE-Nicolet 300 spectrometer. Common reaction solvents were either high performance liquid chromatography (HPLC) grade or American Chemical Society (ACS) grade, and anhydrous as obtained from the manufacturer unless otherwise noted. “Chromatography” or “purified by silica gel” refers to flash column chromatography using silica gel (EM Merck, Silica Gel 60, 230-400 mesh) unless otherwise noted.

### Example 1

### Synthesis of Biaryl Precursors

Scheme 1 depicts the synthesis of various biaryl intermediates useful in producing compounds of the present invention. Known iodoaryl oxazolidinone intermediate 50 (see U.S. Pat. Nos. 5,523,403 and 5,565,571) is coupled to a substituted aryl boronic acid (the Suzuki reaction) to produce biaryl alcohol 51. Mesylate 52, azide 53, and amine 54 are then synthesized using chemistry well known to those skilled in the art. 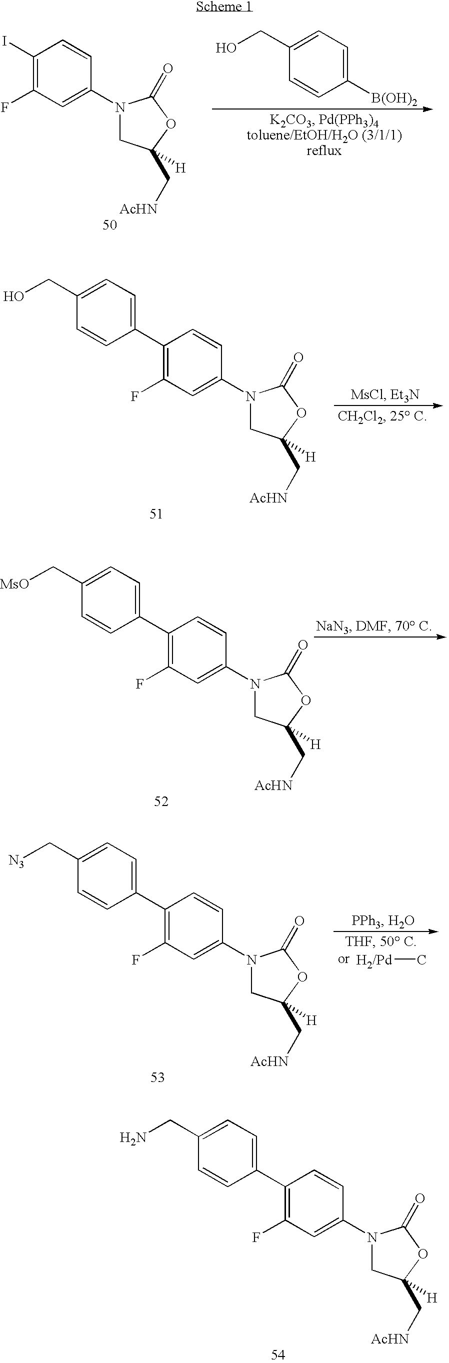
Synthesis of Alcohol 51

A suspension of N-[3-(3-fluoro-4-iodo-phenyl)-2-oxo-oxazolidinyl-5-methyl]-acetamide 50 (14.0 g, 37 mmol) in toluene (120 mL) was treated with 4-(hydroxymethyl)phenylboronic acid (7.87 g, 51.8 mmol, 1.4 equiv), potassium carbonate (K₂CO₃, 15.32 g, 111 mmol, 3.0 equiv), ethanol (EtOH, 40 mL), and H₂O (40 mL) at 25° C., and the resulting mixture was degassed three times under a steady stream of argon at 25° C. Tetrakis(triphenylphosphine)palladium(0) (Pd(PPh₃)₄, 2.14 g, 1.85 mmol, 0.05 equiv) was subsequently added to the reaction mixture, and the resulting reaction mixture was degassed three times before being warmed to gentle reflux for 6 h. When TLC and HPLC showed the coupling reaction was complete, the reaction mixture was cooled to room temperature before being treated with H₂O (240 mL) at room temperature. The resulting mixture was then stirred at room temperature for 10 min before being cooled to 0-5° C. for 1 h. The solid precipitates were collected by filtration, washed with H₂O (2×100 mL) and 20% ethyl acetate (EtOAc)/hexane (2×50 mL), and dried in vacuo. The crude desired N-[3-(2-fluoro-4′-hydroxymethyl-biphenyl-4-yl)-2-oxo-oxazolidin-5-ylmethyl]-acetamide 51 (12.50 g, 94% yield) was obtained as off-white solids. This material was found to be essentially pure by HPLC and ¹H NMR and was directly used in the subsequent reaction without further purification. ¹H NMR (300 MHz, DMSO-d₆) δ 1.76 (s, 3H, COCH₃), 3.35 (t, 2H, J=5.4 Hz), 3.69 (dd, 1H, J=6.4, 9.2 Hz), 4.08 (t, 1H, J=9.1 Hz), 4.46 (d, 2H, J=5.7 Hz, CH₂OH), 4.68 (m, 1H), 5.16 (t, 1H, J=5.7 Hz, OH), 7.25-7.52 (m, 7H, aromatic-H), 8.18 (t, 1H, J=5.8 Hz, NHCOCH₃). LCMS (LSI) m/e 359 (M+H)⁺.

Synthesis of Mesylate 52

A suspension of alcohol 51 (12.49 g, 34.90 mmol) in methylene chloride (CH₂Cl₂, 150 mL) was treated with triethylamine (7.07 g, 9.7 mL, 70 mmol, 2.0 equiv) at 25° C., and the resulting mixture was cooled to 0-5° C. before being treated dropwise with methanesulfonyl chloride (4.80 g, 3.24 mL, 41.9 mmol, 1.2 equiv) at 0-5° C. The resulting reaction mixture was subsequently stirred at 0-5° C. for 2 h. When TLC and HPLC showed the reaction was complete, the reaction mixture was treated with H₂O (100 mL) at 0-5° C. The mixture was then concentrated in vacuo to remove most of the CH₂Cl₂, and the resulting slurry was treated with H₂O (150 mL). The mixture was stirred at room temperature for 10 min before being cooled to 0-5° C. for 30 min. The solid precipitates were collected by filtration, washed with H₂O (2×100 mL) and 20% ethyl acetate/hexane (2×50 mL), and dried in vacuo. The crude desired methanesulfonic acid 4′-[5-(acetylamino-methyl)-2-oxo-oxazolidin-3-yl]-2′-fluoro-biphenyl-4-ylmethyl ester 52 (11.84 g, 78% yield) was obtained as off-white solids. This material was found to be essentially pure by HPLC and ¹H NMR and was directly used in the subsequent reaction without further purification. LCMS (ESI) m/e 437 (M+H)⁺.

Synthesis of Azide 53

A solution of mesylate 52 (9.27 g, 21.26 mmol) in anhydrous N,N-dimethylformamide (DMF, 50 mL) was treated with sodium azide (NaN₃, 5.53 g, 85.04 mmol, 4.0 equiv) at 25° C., and the resulting reaction mixture was warmed to 70-80° C. for 4 h. When TLC and HPLC showed the reaction was complete, the reaction mixture was cooled to room temperature before being treated with H₂O (150 mL) at room temperature. The resulting mixture was stirred at room temperature for 10 min before being cooled to 0-5° C. for 1 h. The solid precipitates were collected by filtration, washed with H₂O (2×100 mL) and 20% ethyl acetate/hexane (2×50 mL), and dried in vacuo. The crude desired N-[3-(4′-azidomethyl-2-fluoro-biphenyl-4-yl)-2-oxo-oxazolidin-5-ylmethyl]-acetamide 53 (7.16 g, 88% yield) was obtained as off-white solids. The material was found to be essentially pure by TLC and HPLC and was directly used in the subsequent reaction without further purification. LCMS (ESI) m/e 384 (M+H)⁺.

Synthesis of Amine 54

A solution of azide 53 (7.16 g, 18.69 mmol) in tetrahydrofuran (THF, 100 mL) was treated with triphenylphosphine (PPh₃, 5.88 g, 22.43 mmol, 1.2 equiv) and H₂O (3.6 g, 3.6 mL, 0.2 mmol, 11.0 equiv) at 25° C., and the resulting reaction mixture was warmed to 50-55° C. for 12 h. When TLC and HPLC showed the reduction reaction was complete, the reaction mixture was cooled to room temperature before the solvents were removed in vacuo. The residue was directly purified by flash column chromatography (0-15% methanol (MeOH)—CH₂Cl₂ gradient elution) to afford the desired N-[3-(4′-aminomethyl-2-fluoro-biphenyl-4-yl)-2-oxo-oxazolidin-5-ylmethyl]-acetamide 54 (5.82 g, 87% yield) as off-white crystals, which was of sufficient purity to be directly used in subsequent reactions. ¹H NMR (300 MHz, DMSO-d₆) δ 1.85 (s, 3H, COCH₃), 3.04 (br. s, 2H, NH₂), 3.44 (t, 2H, J=5.4 Hz), 3.78 (m, 3H), 4.18 (t, 1H, J=9.1 Hz), 4.77 (m, 1H), 7.25-7.60 (m, 7H, aromatic-H), 8.20 (t, 1H, J=5.8 Hz, NHCOCH₃). LCMS (ESI) m/e 359 (M+2H)²⁺.

Scheme 2 depicts the synthesis of N-methyl amine 56, which is useful in producing compounds of the present invention. 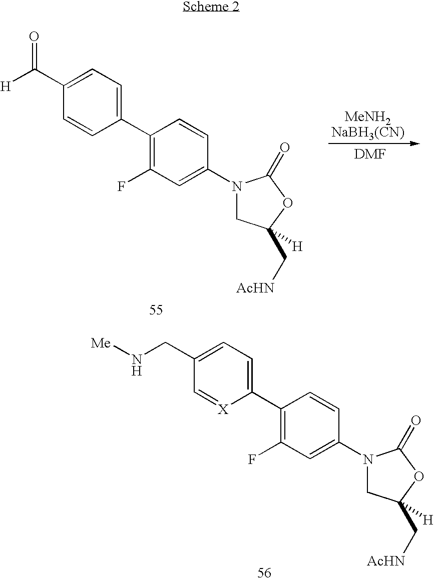
Synthesis of Amine 56 Aldehyde 55 is prepared from iodide 50 and 4-formylboronic acid in the same fashion as alcohol 51 in Example 1 above. A solution of aldehyde 55 (3.56 g, 10.0 mmol) in anhydrous DMF (20 mL) was treated with a 2 N solution of methylamine in THF (25 mL, 50.0 mmol) and sodium triacetoxyborohydride (3.20 g, 15.0 mmol) at room temperature, and the resulting reaction mixture was stirred at room temperature for 6 h. When TLC and LCMS showed that the reaction was complete, the reaction mixture was quenched with H₂O (40 mL), and the resulting mixture was stirred at room temperature for 30 min. The solid precipitate was then collected by filtration, washed with H₂O (2×50 mL), and dried in vacuo. This crude material was subsequently purified by flash column chromatography (5-15% methanol-CH₂Cl₂ gradient elution) to afford amine 56 (2.26 g; 61%) as an off-white solid. ¹H NMR (300 MHz, DMSO-d₆) δ 2.03 (s, 3H, COCH₃), 2.46 (s, 3H, NMe), 3.62 (t, 2H, J=5.4 Hz), 3.86 (s, 2H, Ar—CH₂)), 3.96 (dd, 1H, J=6.4, 9.2 Hz), 4.35 (t, 1H, J=9.2 Hz), 4.90-4.99 (m, 1H), 7.58-7.80 (m, 7H, aromatic-H), 8.45 (t, 1H, J=5.8 Hz, NHCOCH₃). LCMS (ESI) m/z 372 (M+H)⁺.

### Example 2

### Synthesis of Sulfonamide 101

Method A

A suspension of polymeric 4-hydroxy-2,3,5,6-tetrafluorophenol amide resin (TFP resin, J. Comb. Chem. 2000, 2, 691) (1.00 g, 1.27 mmol) in DMF (10 mL) was shaken for 10 minutes in a 70 mL polypropylene cartridge before being treated with ethanesulfonyl chloride (1.02 g, 6.35 mmol) and 4-methylmorpholine (0.60 mL, 7.6 mmol). The reaction mixture was then shaken for 18 h at 23° C. The resin was washed with DMF (10×50 mL), THF (10×50 mL), and methylene chloride (10×50 mL) and dried in vacuo.

A suspension of the above. TFP sulfonate ester (35 mg) in 1 mL of DMF was treated with amine 54 (10 mg, 0.027 mmol) and shaken for 18 h in a 10 mL polypropylene cartridge. The filtrate was collected and dried to give sulfonamide 101 as a solid (15 mg, 0.027 mmol, 100%). ¹HNMR (300 MHz, CD₃CN): δ 7.46-7.30 (m, 6H), 7.22 (dd, J=9, 2 Hz, 1H), 6.52-6.63 (m, 1H), 6.49-6.59 (m, 1H), 4.65-4.57 (m, 1H), 4.14-4.12 (m, 2H), 3.99-3.93 (m, 1H), 3.64 (dd, J=9, 7 Hz, 1H), 3.41-3.38 (m, 2H), 2.86 (dd, J=15, 8 Hz, 2H), 1.14-1.10 (m, 3H). LCMS (ESI): m/e 449.7 (M+H)⁺.

Method B

A solution of amine 54 (0.10 g, 0.28 mmol) in methylene chloride (1.6 mL) at 0° C. was treated with triethylamine (0.057 mL, 0.56 mmol) and ethanesulfonyl chloride (0.030 mL, 0.31 mmol). The reaction mixture was warmed to 23° C. and stirred for 2 h. Additional ethanesulfonyl chloride (0.005 mL, 0.05 mmol) was added, and the reaction mixture was stirred for 1 h. The reaction mixture was diluted with methylene chloride and washed with 1 M hydrochloric acid and saturated aqueous sodium bicarbonate. Drying over sodium sulfate (Na₂SO₄) and evaporation of the solvent yielded crude product, which was purified by flash chromatography (1:4.5:4.5 methanol/methylene chloride/ethyl acetate) to afford sulfonamide 101 (0.062 g, 0.14 mmol, 50%). MS (ESI: 450 (M+H)⁺.

### Example 3

### Synthesis of Sulfonamide 102

A solution of amine 56 (0.085 g, 0.23 mmol) in THF (1.2 mL) and water (1.2 mL) was treated with 1 M aqueous sodium hydroxide (0.026 mL, 0.026 mmol) and ethanesulfonyl chloride (0.024 mL, 0.25 mmol) and stirred at 23° C. for 15 minutes. The reaction mixture was diluted with methylene chloride (20 mL) and washed with 1 M hydrochloric acid (20 mL) and saturated aqueous sodium bicarbonate (20 mL). Drying over Na₂SO₄ and evaporation of solvent yielded crude product, which was purified by flash chromatography (4.5:4.5:1 methylene chloride/ethyl acetate/methanol) to afford sulfonamide 102 (0.050 g, 0.11 mmol, 48%). MS (ESI): 527 (M+Na+CH₃CN)⁺.

### Example 4

### Synthesis of Sulfonamide 103

A solution of amine 54 (0.085 g, 0.24 mmol) in THF (1.2 mL) and water (1.2 mL) was treated with 1 M aqueous sodium hydroxide (0.026 mL, 0.026 mmol) and 1-propanesulfonyl chloride (0.029 mL, 0.26 mmol) and stirred at 23° C. for 1 h The reaction mixture was diluted with methylene chloride (20 mL) and washed with 1 M hydrochloric acid (20 mL) and saturated aqueous sodium bicarbonate (20 mL). Drying over Na₂SO₄ and evaporation of solvent yielded crude product, which was purified by flash chromatography (4.5:4.5:1 methylene chloride/ethyl acetate/methanol) to afford sulfonamide 103 (0.040 g, 0.086 mmol, 36%). MS (ESI): 357 (M+Na+CH₃CN)⁺.

### Example 5

### Synthesis of Sulfonamide 104

A solution of amine 54 (0.085 g, 0.24 mmol) in DMF (2.4 mL) was treated with triethylamine (0.066 mL, 0.48 mmol) and methanesulfonyl chloride (0.020 mL, 0.26 mmol) and stirred at 23° C. for 1 h. Additional methanesulfonyl chloride (0.004 mL, 0.048 mmol) was then added, and the reaction mixture stirred for 2 h. Additional methanesulfonyl chloride (0.004 mL, 0.048 mmol) was then added, and the reaction mixture stirred for 5 minutes. The reaction mixture was diluted with ethyl acetate (20 mL) and washed with 1 M hydrochloric acid (20 mL) and saturated aqueous sodium bicarbonate (20 mL). Drying over Na₂SO₄ and evaporation of solvent yielded crude product, which was purified by flash chromatography (2.5-10% methanol in methylene chloride) to afford sulfonamide 104 (0.047 g, 0.11 mmol, 46%). MS (ESI): 436 (M+H)⁺.

### Example 6

### Synthesis of Sulfonamide 105

A solution of amine 54 (0.085 g, 0.24 mmol) in methylene chloride (2.4 mL) was treated with triethylamine (0.066 mL, 0.48 mmol) and 2-chloroethanesulfonyl chloride (0.027 mL, 0.26 mmol) and stirred at 23° C. After 1.5 h, additional triethylamine (0.066 mL, 0.48 mmol) and 2-chloroethanesulfonyl chloride (0.024 mL, 0.24 mmol) were added. The reaction mixture was diluted with methylene chloride (20 mL) and washed with 1 M hydrochloric acid (20 mL) and th saturated aqueous sodium bicarbonate (20 mL). Drying over Na₂SO₄ and evaporation of solvent yielded sulfonamide 105 (0.044 g, 0.098 mmol, 41%). MS (ESI): 511 (M+Na+CH₃CN)⁺.

### Example 7

### Synthesis of Sulfonamide 106

A solution of amine 54 (0.070 mg, 0.20 mmol) in DMF (1.0 mL) was treated with triethylamine (0.055 mL, 0.40 mmol) and chloromethanesulfonyl chloride (0.019 mL, 0.22 mmol) and stirred at 23° C. for 16 h. Additional chloromethanesulfonyl chloride (0.009 mL, 0.10 mmol) was added to the reaction mixture as a 0.2 M solution in DMF, and the reaction mixture was stirred for 1 h. The reaction mixture was cooled to −40° C., and additional chloromethanesulfonyl chloride (0.017 mL, 0.20 mmol) was added, followed by stirring at 23° C. for 1 h. The reaction mixture was diluted with methylene chloride (20 mL) and washed with 1 M hydrochloric acid (20 mL) and saturated aqueous sodium bicarbonate (20 mL). Drying over Na₂SO₄ and evaporation of solvent yielded crude product, which was purified by flash chromatography (4.5:4.5:1 methylene chloride/ethyl acetate/methanol) to afford sulfonamide 106 (0.048 g, 0.10 mmol, 50%). MS (ESI): 533 (M+Na+CH₃CN)⁺.

### Example 8

### Synthesis of Sulfonamide 107

Scheme 3 depicts the synthesis of sulfonamide 107.

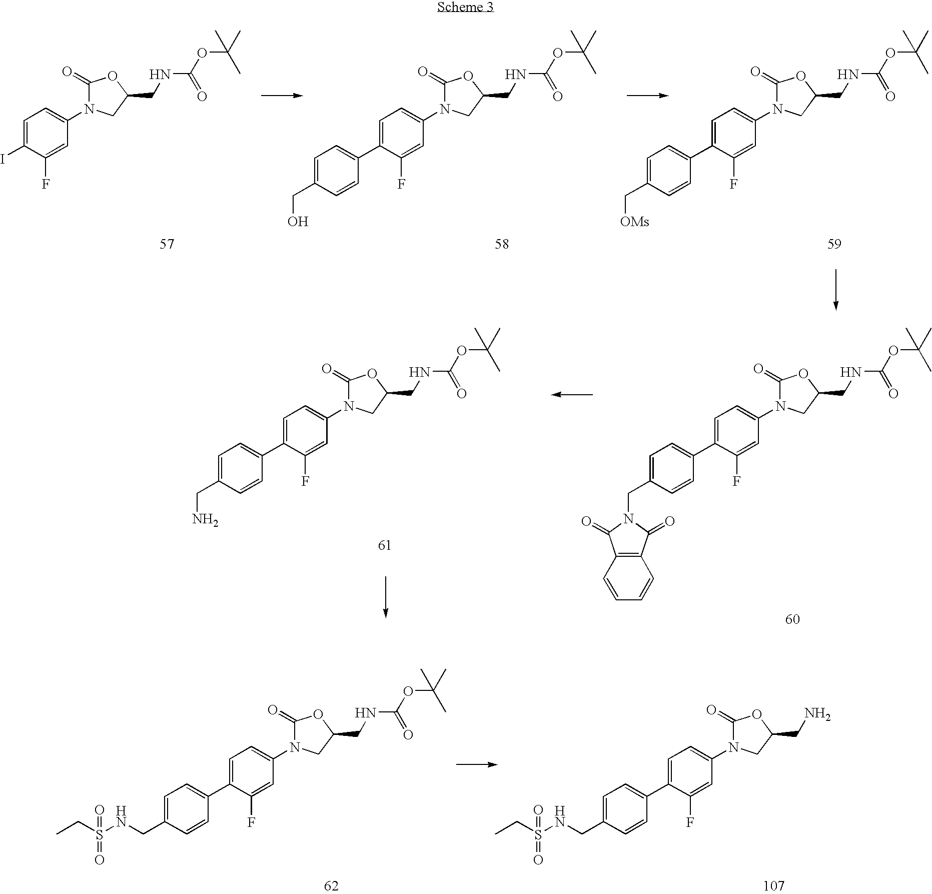

A solution of iodoarene 57 (1.2 g, 2.6 mmol) in toluene (9 mL), water (3 mL), and ethanol (6 mL) was treated with potassium carbonate (1.1 g, 7.8 mmol), 4-hydroxymethylphenyl boronic acid (0.43 g, 3.1 mmol), and Pd(PPh₃)₄ (0.11 g, 0.13 mmol) and heated to reflux for 5 h. The reaction mixture was then cooled to 23° C., diluted with ethyl acetate (50 mL) and washed with water (50 mL). Drying over Na₂SO₄ and evaporation yielded crude product, which was purified by flash chromatography (40-80% ethyl acetate in hexane) to afford alcohol 58 as a solid (0.62 g, 1.5 mmol, 58%).

A solution of alcohol 58 (0.62 g, 1.5 mmol) in methylene chloride (7.5 mL) was cooled to 0° C. and treated with triethylamine (420 mL, 3.0 mmol) and methanesulfonyl chloride (132 mL, 1.7 mmol). The reaction mixture then was warmed to 23° C. and stirred for 10 minutes. The reaction mixture was diluted with methylene chloride (50 mL) and washed with 1 M hydrochloric acid (30 mL) and saturated aqueous sodium bicarbonate (30 mL). Drying over Na₂SO₄ and evaporation of solvent afforded mesylate 59 as a solid (0.69 g, 1.4 mmol, 93%).

A solution of mesylate 59 (0.68 g, 1.4 mmol) in DMF (7.6 mL) was treated with potassium phthalimide (0.28 g, 1.5 mmol) and stirred at 80° C. for 3 h. The reaction mixture then was stirred at 23° C. for 12 h. The reaction mixture was poured into an ice and water mixture (50 mL) and the precipitate was recovered via vacuum filtration to afford phthalimide 60 as a white foam (0.69 g, 1.3 mmol, 93%).

A solution of phthalimide 60 (0.62 g, 1.1 mmol) in ethanol (5.5 mL) was treated with hydrazine monohydrate (0.27 mL, 5.5 mmol) and stirred at 70° C. for 1.5 h. The reaction mixture was diluted with water (25 mL) and extracted with methylene chloride (30 mL). The organic layer was washed with water (2×30 mL), dried over Na₂SO₄, and the solvent removed in vacuo to afford amine 61 as a brown oil (0.53 mg). This crude material was directly taken on to the next reaction.

A solution of amine 61 (0.48 g, 1.2 mmol) in methylene chloride (6.0 mL) was treated with triethylamine (0.34 mL, 2.4 mmol) and ethanesulfonylchloride (0.12 mL, 1.3 mmol) and stirred at 23° C. for 2 h. The reaction mixture was diluted with methylene chloride (50 mL) and washed with 1 M hydrochloric acid (50 mL) and saturated aqueous sodium bicarbonate (50 mL). Drying over Na₂SO₄ and evaporation of solvent yielded crude product, which was purified by flash chromatography (20-40% ethyl acetate in hexane) to afford sulfonamide 62 as a white powder (0.45 g, 0.89 mmol, 74%).

A solution of sulfonamide 62 (0.050 g, 0.099 mmol) in methylene chloride (0.50 mL) was treated with 4.0 M hydrogen chloride in dioxane (0.50 mL) and stirred at 23° C. for 16 h, when additional 4.0 M hydrogen chloride in dioxane (0.50 mL) was added and the reaction mixture stirred for 1 h. The solvent was removed in vacuo to yield sulfonamide 107 as the hydrochloride salt (0.041 g, 0.091 mmol, 92%). MS (ESI): 449 (M+H+CH₃CN)⁺.

### Example 9

### Synthesis of Sulfonamide 108

A solution of sulfonamide 107 (0.050 g, 0.11 mmol) in methanol (0.10 mL) was treated with formaldehyde (37% by weight in water, 0.061 mL, 0.75 mmol), acetic acid (0.020 mL, 0.34 mmol), and sodium triacetoxyborohydride (0.12 g, 0.57 mmol) and stirred at 23° C. for 15 minutes. The reaction mixture was diluted with methylene chloride (10 mL) and washed with saturated aqueous sodium bicarbonate (10 mL). Drying over Na₂SO₄ and evaporation of solvent yielded crude product, which was purified by flash chromatography (4.5:4.5:1 methylene chloride/ethyl acetate/methanol) to afford sulfonamide 108 (0.040 g, 0.092 mmol, 84%). MS (ESI): 477 (M+H+CH₃CN)⁺.

### Example 10

### Synthesis of Sulfonamide 109

Scheme 4 depicts the synthesis of sulfonamide 109 from sulfonamide 107.

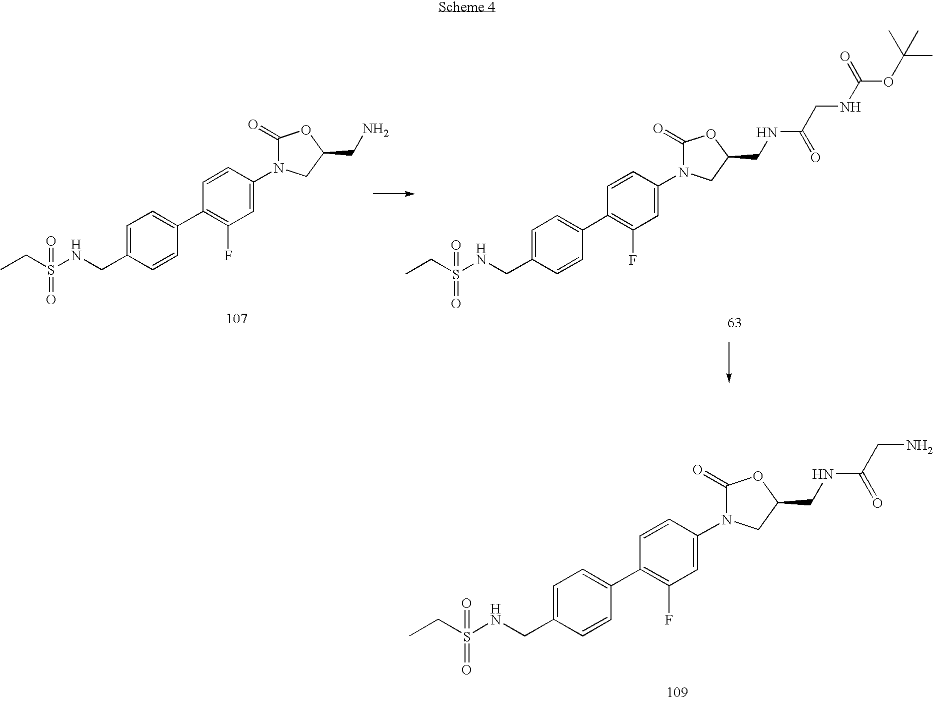

A solution of sulfonamide 107 (0.050 g, 0.11 mmol) in methylene chloride (4.0 mL) was treated with diisopropylethyl amine-(0.039 mL, 0.23 mmol), N-Boc-glycine (0.020 g, 0.11 mmol), and 1-(3-dimethylaminopropyl)-3-ethylcarbodiimide hydrochloride (EDCI, 0.024 g, 0.12 mmol) and stirred at 23° C. After 1 h, additional DMF (2 mL), EDCI (0.024 g, 0.12 mmol), and N-Boc-glycine (0.010 g, 0.057 mmol) were added to the reaction mixture. After 16 h, additional EDCI (0.024 g, 0.12 mmol), and N-Boc-glycine (0.010 g, 0.057 mmol) were added to the reaction mixture. After 2 h, diisopropylethylamine (0.20 mL, 1.1 mmol), EDCI (0.043 g, 0.22 mmol), and N-Boc-glycine (0.020 g, 0.11 mmol) were added to the reaction mixture with stirring for 1 h at 23° C. The reaction mixture was diluted with methylene chloride (30 mL) and washed with saturated aqueous sodium bicarbonate. Drying over Na₂SO₄ and evaporation of solvent yielded amide 63 (0.023 g, 0.041 mmol) as crude product, which was taken on directly to the next reaction.

A solution of amide 63 (0.023 g, 0.041 mmol) was treated with 4.0 M hydrogen chloride in dioxane (0.20 mL) and stirred at 23° C. for 1 h. The solvent was removed in vacuo to yield sulfonamide 109 as the hydrochloride salt (0.015 g, 0.030 mmol, 73%). MS (ESI): 465 (M+H)⁺.

### Example 11

### Synthesis of Sulfonamide 110

Scheme 5 depicts the synthesis of sulfonamide 110 from amine 61.

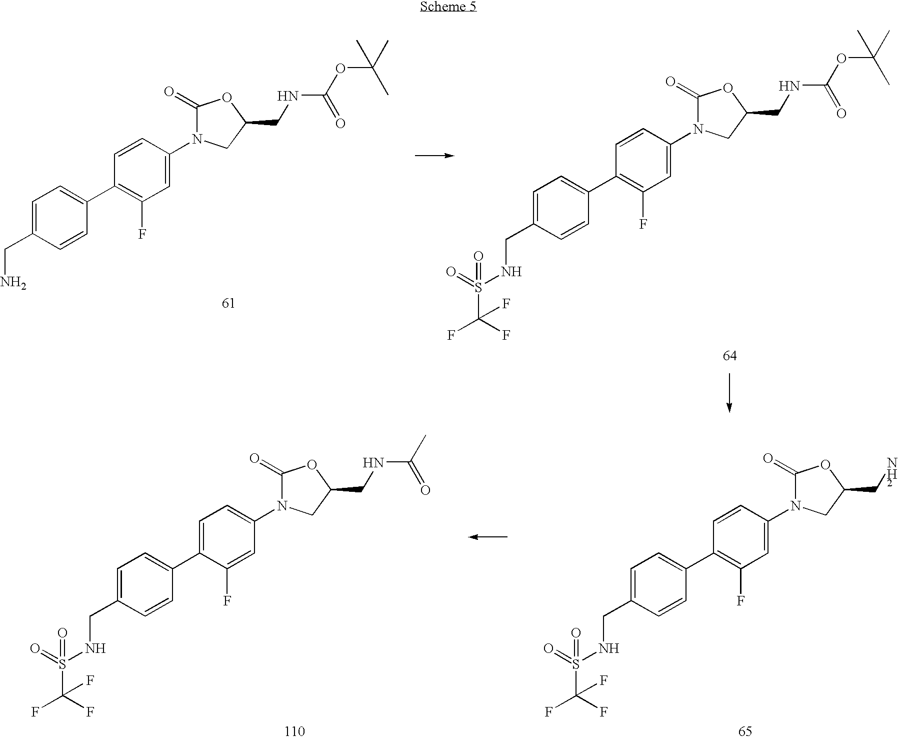

A solution of amine 61 (0.050 g, 0.12 mmol) in methylene chloride (2.5 mL) and triethylamine (0.63 mL) was cooled to −78° C. and treated with trifluoromethanesulfonic anhydride (0.022 mL, 0.13 mmol). The reaction mixture was stirred for 15 minutes before being diluted with methylene chloride (30 mL) and washed with 1 M hydrochloric acid (20 mL). Drying over Na₂SO₄ and evaporation of solvent afforded sulfonamide 64 (0.050 g, 0.091 mmol, 76%).

A solution of sulfonamide 64 (0.050 g, 0.091 mmol) in 4.0 M hydrogen chloride in dioxane (0.45 mL) was stirred at 23° C. for 1 h. The solvent was removed in vacuo, and the reaction mixture was four times diluted with methylene chloride and the solvent evaporated to yield amine 65 as the hydrochloride salt (0.040 g, 0.083 mmol, 91%). MS (ESI): 448 (M+H)⁺.

A solution of amine 65 (0.040 g, 0.083 mmol) in methylene chloride (0.42 mL) was cooled to 0° C. and treated with triethylamine (0.023 mL, 0.17 mmol) and acetic anhydride (0.009 mL, 0.091 mmol), followed by stirring for 15 minutes. The reaction mixture was diluted with methylene chloride (15 mL) and washed with 1 M hydrochloric acid (10 mL). Drying over Na₂SO₄ and evaporation of solvent afforded sulfonamide 110 (16 mg, 0.033 mmol, 40%). MS (ESI): 490 (M+H)⁺.

### Example 12

### Synthesis of Sulfonamide 111

Scheme 6 depicts the synthesis of sulfonamide 111 from azide 53.

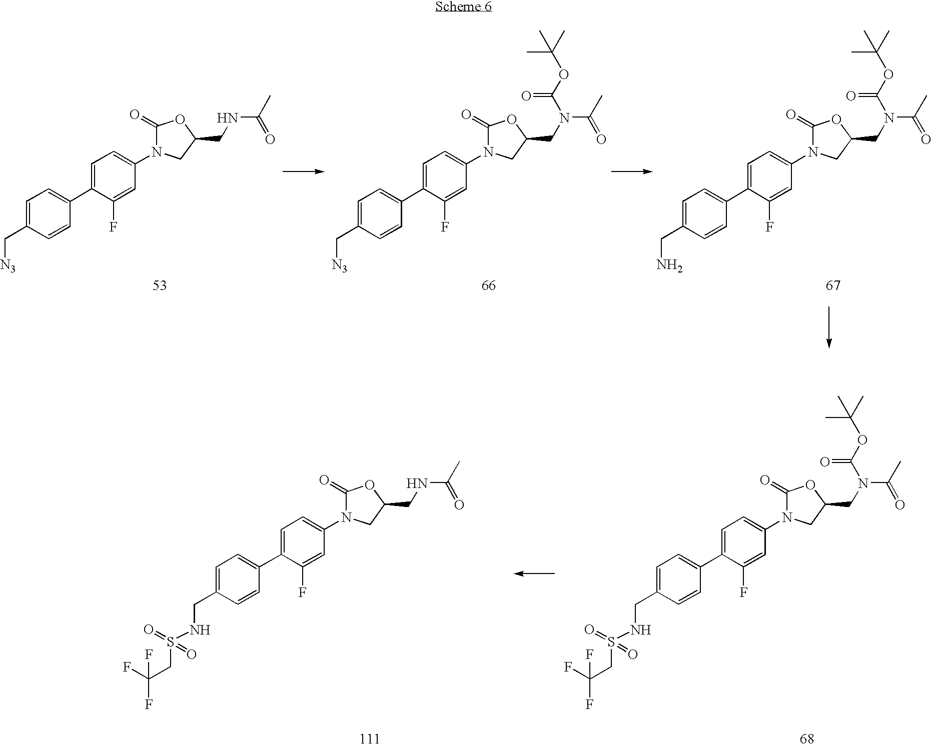

A solution of azide 53 (0.38 g, 0.99 mmol) in methylene chloride (0.99 mL) was treated with triethylamine (0.14 mL, 0.99 mmol), ditertbutyldicarbonate (0.26 mL, 1.2 mmol), and 4-dimethylaminopyridine (DMAP, 0.12 g, 0.99 mmol) and stirred at 23° C. for 1 h. The reaction mixture was diluted with methylene chloride (20 mL) and washed with saturated aqueous sodium bicarbonate (15 mL). Drying over Na₂SO₄ and evaporation yielded crude product, which was purified by flash chromatography (0-5% ethyl acetate in methylene chloride) to afford BOC-acetamide 66 as a white foam (0.36 g, 0.75 mmol, 76%).

A solution of BOC-acetamide 66 (0.36 g, 0.75 mmol) in methanol (10 mL) and THF (2 mL) was treated with 20% palladium on charcoal (60 mg, 5%), flushed with hydrogen gas three times, and stirred at 23° C. for 16 h. The reaction mixture was filtered through Celite to afford amine 67 (0.35 g). This crude material was directly used in the next reaction. MS (ESI): 458 (M+H)⁺.

A solution of amine 67 (0.070 g, 0.15 mmol) in methylene chloride (3.2 mL) and triethylamine (0.80 mL) was cooled to −78° C. and treated with trifluoroethylsulfonyl chloride (0.031 mL, 0.17 mmol) and stirred for 1 h. The reaction mixture was diluted with methylene chloride (25 mL) and washed with 1 M hydrochloric acid (20 mL) and saturated aqueous sodium bicarbonate (20 mL). Drying over Na₂SO₄ and evaporation of solvent yielded crude product, which was purified by flash chromatography (methylene chloride) to afford sulfonamide 68 (0.031 g, 0.051 mmol, 34%). MS (ESI): 667 (M+Na+CH₃CN)⁺.

A solution of sulfonamide 68 (0.031 g, 0.051 mmol) in trifluoroacetic acid (0.20 mL) was stirred at 23° C. for 15 minutes. The solvent was removed in vacuo, and the reaction mixture was four times diluted with methylene chloride and the solvent evaporated to afford sulfonamide 111 (0.014 g, 0.028 mmol, 55%). MS (ESI): 504 (M+H)⁺.

### Example 13

### Synthesis of Sulfonamide 112

Scheme 7 depicts the synthesis of sulfonamide 112 from amine 67.

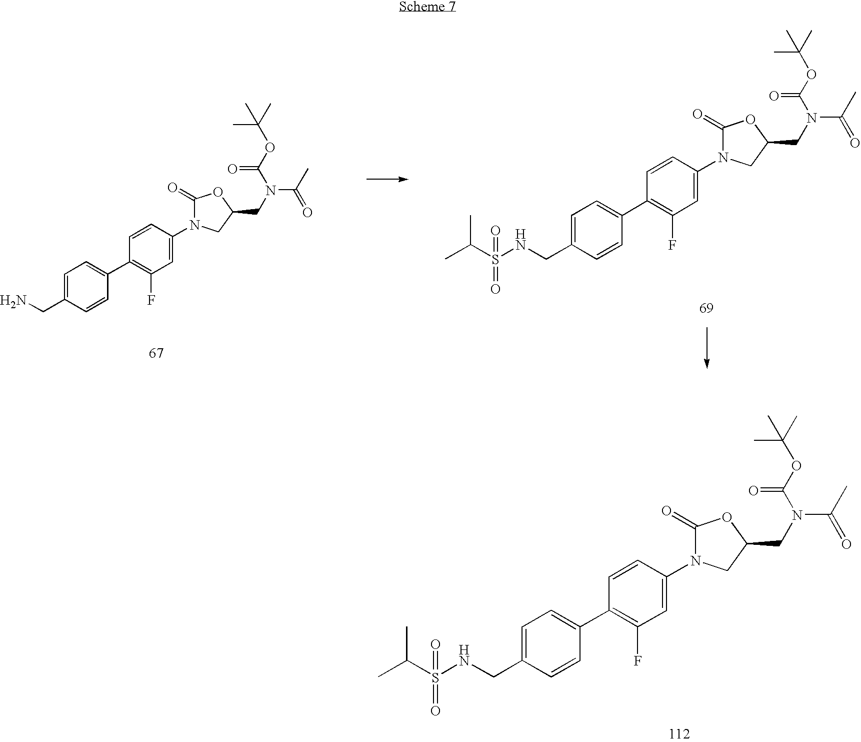

A solution of amine 67 (0.070 g, 0.15 mmol) in methylene chloride (3.2 mL) and triethylamine (0.80 mL) was cooled to −78° C., treated with isopropylsulfonyl chloride (0.024 mL, 0.17 mmol) and stirred for 30 minutes at 23° C. The reaction mixture was diluted with methylene chloride (40 mL) and washed with 1 M hydrochloric acid (40 mL) and saturated aqueous sodium bicarbonate (40 mL). Drying over Na₂SO₄ and evaporation of solvent yielded crude product, which was purified by flash chromatography (10-20% ethyl acetate in chloroform) to afford sulfonamide 69 (0.031 g, 0.055 mmol, 37%).

A solution of sulfonamide 69 (0.031 mg, 0.055 mmol) was treated with trifluoroacetic acid (0.28 mL) and stirred at 23° C. for 10 minutes. The solvent was removed in vacuo to yield sulfonamide 112 as the hydrochloride salt (0.027 g, 0.055 mmol, 100%). MS (ESI): 527 (M+Na+CH₃CN)⁺.

### Example 14

### Synthesis of Sulfonamide 113

Scheme 8 depicts the synthesis of sulfonamide 113 from carboxylic acid 70.

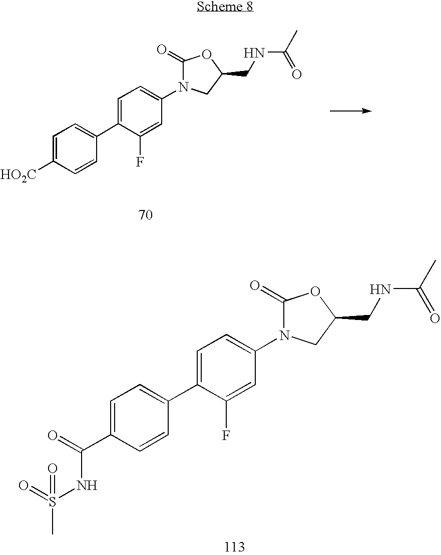

A suspension of 0.050 g (0.13 mmol) of carboxylic acid 70 (made from iodide 50 and 4-carboxyphenyl boronic acid in the same fashion as alcohol 51 in Example 1) in DMF (1.5 mL) was treated with methyl sulfonamide (0.013 g, 0.13 mmol), EDCI (0.038 g, 0.20 mmol) and DMAP (0.024 g, 0.20 mmol), and stirred at 23° C. for 12 h. Additional amounts of methyl sulfonamide (0.039 g, 0.4 mmol), EDCI (0.076 g, 0.40 mmol), and DMAP (0.024 g, 0.20 mmol) were added and the mixture stirred at 23° C. for an additional 72 h. The reaction mixture was diluted with methylene chloride (30 mL) and methanol (15 mL), washed with 1 M hydrochloric acid (2×20 mL), and evaporated. Purification by preparative TLC (10% methanol, 45% ethyl acetate, 45% methylene chloride) afforded sulfonamide 113 (0.040 g, 0.089 mmol, 66%) as a white powder. MS (ESI): 450 (M+H)⁺.

### Example 15

### Synthesis of Sulfonamide 114

Scheme 9 depicts the synthesis of sulfonamide 114 from amine 71.

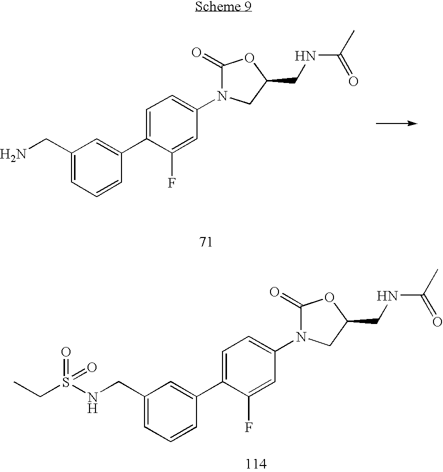

A solution of 0.10 g (0.25 mmol) of amine hydrochloride 71 (made from iodide 50 and 3-(BOC-methylamine)phenyl boronic acid in the same fashion as alcohol 51 in Example 1, followed by BOC deprotection) in methylene chloride (3 mL) at 0° C. was treated with triethylamine (0.11 mL, 0.76 mmol) and ethanesulfonyl chloride (0.035 mL, 0.38 mmol). The solution then was warmed to 23° C. and stirred for 1 h. The reaction mixture was diluted with methylene chloride (50 mL) and methanol (10 mL), washed with 1 M hydrochloric acid (2×25 mL), dried over Na₂SO₄, and evaporated. Purification by preparative TLC (10% methanol, 90% methylene chloride) afforded sulfonamide 114 (0.095 g, 0.021 mmol, 85%) as a white powder. MS (ESI): 450 (M+H)⁺.

### Example 16

### Synthesis of Sulfonamide 115

A solution of amine hydrochloride 71 (0.10 g, 0.25 mmol) in methylene chloride (3 mL) at 0° C. was treated with triethylamine (0.11 mL, 0.76 mmol) and metahnesulfonyl chloride (0.029 mL, 0.38 mmol). The solution was then warmed to 23° C. and stirred for 0.5 h. The reaction mixture was diluted with methylene chloride (50 mL) and methanol (5 mL), washed with 1 M hydrochloric acid (2×25 mL), dried over Na₂SO₄, and evaporated. Purification by preparative TLC (10% methanol, 90% methylene chloride) afforded sulfonamide 115 (0.085 g, 0.019 mmol, 78%) as a white powder. MS (ESI): 436 (M+H)⁺.

### Example 17

### Synthesis of Sulfonamide 116

Scheme 10 depicts the synthesis of sulfonamide 116 from amine 72.

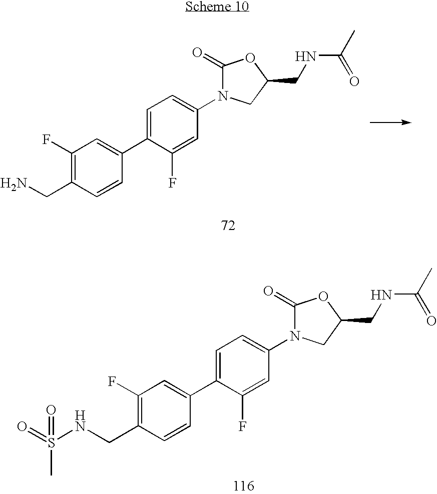

Amine 72 was prepared by coupling iodide 50 and 3-fluoro-4-formylphenyl boronic acid, followed by reduction with sodium borohydride. The resulting alcohol was then converted to a mesylate, azide, and ultimately amine 72 in the same fashion as amine 54 in Example 1. A solution of amine 72 (0.093 g, 0.25 mmol) in DMF (1.5 mL) was treated with triethylamine (0.10 mL, 0.75 mmol) and metahnesulfonyl chloride (0.029 mL, 0.38 mmol) and stirred for 0.5 h. The reaction mixture was diluted with methylene chloride (50 mL), washed with 1 M hydrochloric acid (2×20 mL), dried over Na₂SO₄, and evaporated. Purification by preparative TLC (10% methanol, 90% methylene chloride) afforded sulfonamide 116 (0.095 g, 0.021 mmol, 84%) as a white powder. MS (ESI): 454 (M+H)⁺.

### Example 18

### Synthesis of Sulfonamide 117

Scheme 11 depicts the synthesis of sulfonamide 117 from amine 54.

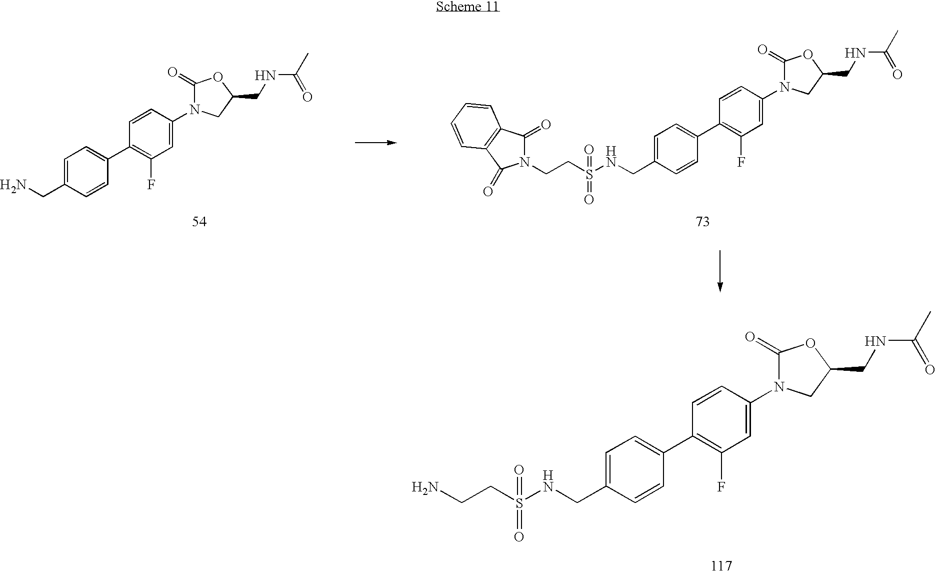

A solution of amine 54 (0.070 g, 0.20 mmol) in DMF (1.0 mL) was treated with triethylamine (0.055 mL, 0.40 mmol) and 2-phthalimidoethanesulfonyl chloride (0.059 mg, 0.22 mmol) and stirred at 23° C. for 3.5 h. Additional 2-phthalimidoethanesulfonyl chloride (0.081 mg, 0.30 mmol) and triethylamine (0.087 mL, 0.63 mmol) were added, and the reaction mixture was stirred for 16 h. The reaction mixture was diluted with methylene chloride (20 mL) and washed with 1 M hydrochloric acid (20 mL) and saturated aqueous sodium bicarbonate (20 mL). Drying over Na₂SO₄ and evaporation of solvent yielded crude product, which was purified by flash chromatography (2.5-5% methanol in 1:1 methylene chloride/ethyl acetate) to afford phthalimide 73 (0.082 g, 0.14 mmol, 70%). MS (ESI): 617 (M+Na)⁺.

A solution of phthalimide 73 (0.065 g, 0.11 mmol) in ethanol (0.55 mL) was treated with hydrazine monohydrate (0.026 mL, 0.55 mmol) and stirred at 70° C. for 1 h. The reaction mixture was cooled to 23° C. and diluted with water (50 mL). The water layer was extracted with methylene chloride (50 mL), and the organic layer was washed twice with water (50 mL). Drying over Na₂SO₄ and evaporation of solvent yielded crude product, which was purified by flash chromatography (4.5:4.5:1 methylene chloride/ethyl acetate/methanol) to afford sulfonamide 117 (0.015 g, 0.032 mmol, 29%). MS (ESI): 465 (M+H)⁺.

### Example 19

### Synthesis of Sulfonamide 118

Scheme 12 depicts the synthesis of (4-bromo-phenyl)-methanesulfonyl chloride 78, a precursor to sulfonamide 118.

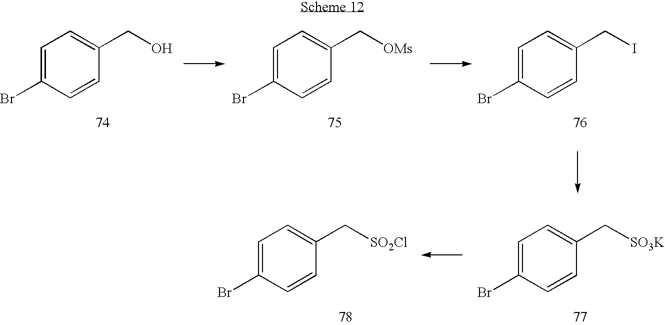

To a stirred 0° C. mixture of p-bromobenzyl alcohol 74 (7.4 g, 40 mmol), triethylamine (8.3 mL) and CH₂Cl₂ (100 mL) was added methane sulfonyl chloride (3.2 mL, 41.5 mmol). The solution was allowed to warm to room temperature and stirred for 16 h. The reaction mixture was poured into 100 mL saturated aqueous sodium bicarbonate (NaHCO₃) and extracted with CH₂Cl₂ (2×50 mL). The combined organic extracts were dried over Na₂SO₄, filtered, and concentrated to give 8.2 g of mesylate 75 as a yellow oil. This oil was dissolved in 50 mL acetone and 10 g of sodium iodide was added. The reaction mixture refluxed for 1 h. After cooling to room temperature, the reaction mixture was diluted with water (300 mL) and extracted with 3:1 hexane/ether mixture (3×100 mL). The combined organic extracts were washed with brine, dried over magnesium sulfate (MgSO₄), filtered, and concentrated to yield 8.6 g of iodide 76 as a white solid. This solid was redissolved in acetone (25 mL) 26 mL of 1N sodium sulfite (Na₂SO₃) was added followed by 14 g potassium carbonate (K₂CO₃). This suspension was heated in a sealed tube in a 90° C. oil bath for 1.5 h. The reaction mixture was cooled and poured ether (400 mL). The ether suspension was stirred for 2 h, and the solids were filtered and rinsed with water (2×50 mL) and ether (2×50 mL) and finally dried overnight in vacuo to afford 8.1 g of salt 77 as a white solid. This solid was suspended in CH₂Cl₂ (50 mL) and cooled to −20° C. Oxalyl chloride (4 mL) was added by syringe over 5 minutes followed by three drops of DMF. The reaction mixture was stirred at −20° C. for 30 minutes then allowed to warm to room termperature and stirred for 2 h. The reaction mixture was diluted to 150 mL with CH₂Cl₂, washed with water and brine, dried over Na₂SO₄, filtered and concentrated to afford methanesulfonyl chloride 78 as a yellow oil (7.8 g).

Scheme 13 depicts the synthesis of sulfonamide 118 from methanesulfonyl chloride 78.

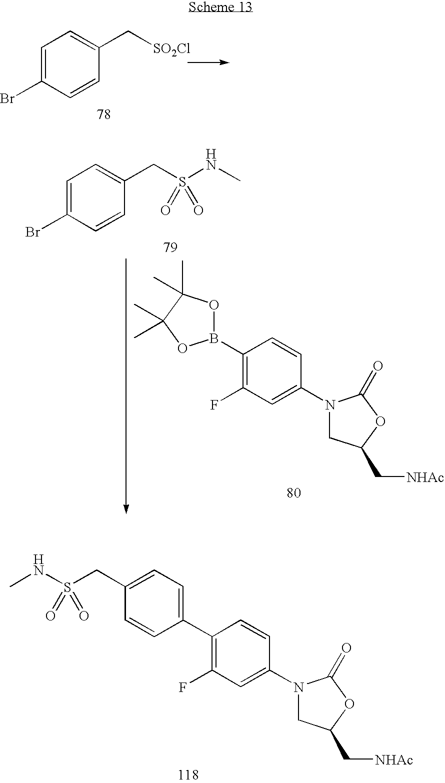

To a stirred solution of methanesulfonyl chloride 78 (0.3 g, 1.11 mmol) in CH₂Cl₂ (2 mL) was added methylamine (200 uL). The mixture was stirred for 1 h, then diluted with 20 mL CH₂Cl₂ and washed with saturated aqueous NaHCO₃ and brine. The organic fraction was dried over K₂CO₃, filtered and concentrated to give 0.24 g of amine 79 as a tan solid which was used without further purification.

A suspension of N-[3-(3-fluoro-4-iodo-phenyl)-2-oxo-oxazolidin-5-ylmethyl]-acetamide 50 (20.0 g, 52.8 mmol) in anhydrous 1,4-dioxane (130 mL) was treated with 4,4,5,5-tetramethyl-[1,3,2]dioxaborolane (10.2 g, 11.6 mL, 80.0 mmol) and triethylamine (16.0 g, 22.4 mL, 158.4 mmol) at room temperature. The resulting reaction mixture was degassed three times under a steady stream of argon before being treated with dichloro[1,1′-bis(diphenylphosphino)ferrocene]palladium (II) (Pd(dppf)₂Cl₂, 1.32 g, 1.6 mmol, 0.03 equiv) at room temperature. The reaction mixture was then degassed three times again under a steady stream of argon before being heated to reflux for 7 h. When TLC and LCMS showed that the reaction was complete, the reaction mixture was cooled to room temperature before being treated with water (100 mL) and ethyl acetate (100 mL). The two layers were separated, and the aqueous layer was extracted with ethyl acetate (2×50 mL). The combined organic extracts were washed with water (2×50 mL) and brine (50 mL), dried over MgSO₄, and concentrated in vacuo. The residual brown oil was further dried in vacuo to afford the crude desired N-{3-[3-fluoro-4-(4,4,5,5-tetramethyl-[1,3,2]dioxaborolan-2-yl)-phenyl]-2-oxo-oxazolidin-5-ylmethyl}acetamide 80 (18.8 g, 20.0 g theoretical, 94%) as a brown solid which was of sufficient purity to be used in subsequent reactions.

A mixture of 95 mg of amine 79 (0.36 mmol), 162 mg of boronic ester 80 (0.43 mmol), 21 mg Pd(PPh₃)₄ (0.018 mmol), and K₂CO₃, (149 mg, 1.1 mmol) was degassed and dissolved in 5 mL of a 3:1:1 toluene/ethanol/water mixture. This solution was heated to 80° C. in a sealed tube for 3 h then cooled to 0° C. The solids were filtered and recrystallized from hot methanol to afford 81 mg of N-[3-(2-fluoro-4′-methylsulfamoylmethyl-biphenylyl)-2-oxo-oxazolidin-5-ylmethyl]-acetamide 118. LCMS (ESI) m/z 477.2 (M+CH₃CN+H)⁺.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound having the formula: 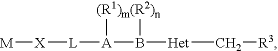
or a pharmaceutically acceptable salt, ester or prodrug thereof, wherein:
A is selected from the group consisting of:
phenyl, pyridyl, pyrazinyl, pyrimidinyl, and pyridazinyl;
B is selected from the group consisting of:
phenyl, pyridyl, pyrazinyl, pyrimidinyl, and pyridazinyl;
Het-CH₂—R³ is selected from the group consisting of: 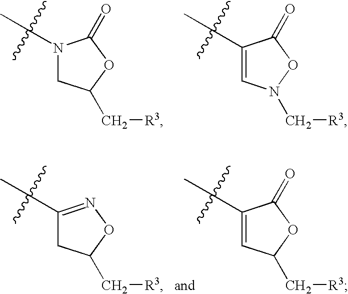
M is selected from the group consisting of:
a) C₁₋₆ alkyl, b) C₂₋₆ alkenyl, and c) C₂₋₆ alkynyl,
wherein any of a)-c) optionally is substituted with one or more R⁴ groups;
X is selected from the group consisting of:
a) —SO₂NR⁴—, and b) —NR⁴SO₂—;
L is selected from the group consisting of:
a) C₁₋₆ alkyl, b) C₂₋₆ alkenyl, and c) C₂₋₆ alkynyl,
wherein any of a)-c) optionally is substituted with one or more R⁴ groups;
R¹, at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR⁷C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —NR⁷C(NR⁷)NR⁷R⁷, aa) —S(O)ₚR⁷, bb) —SO₂NR⁷R⁷, and cc) R⁷;
R², at each occurrence, independently is selected from the group consisting of:
a) F, b) Cl, c) Br, d) I, e) —CF₃, f) —OR⁷, g) —CN, h) —NO₂, i) —NR⁷R⁷, j) —C(O)R⁷, k) —C(O)OR⁷, l) —OC(O)R⁷, m) —C(O)NR⁷R⁷, n) —NR⁷C(O)R⁷, o) —OC(O)NR⁷R⁷, p) —NR C(O)OR⁷, q) —NR⁷C(O)NR⁷R⁷, r) —C(S)R⁷, s) —C(S)OR⁷, t) —OC(S)R⁷, u) —C(S)NR⁷R⁷, v) —NR⁷C(S)R⁷, w) —OC(S)NR⁷R⁷, x) —NR⁷C(S)OR⁷, y) —NR⁷C(S)NR⁷R⁷, z) —NR⁷C(NR⁷)NR⁷R⁷, aa) —S(O)ₚR⁷, bb) —SO₂NR⁷R⁷, and cc) R⁷;
R³ is selected from the group consisting of:
a) —OR⁷, b) —NR⁷R⁷, c) —C(O)R⁷, d) —C(O)OR⁷, e) —OC(O)R⁷, f) —C(O)NR⁷R⁷, g) —NR⁷C(O)R⁷, h) —OC(O)NR⁷R⁷, i) —NR⁷C(O)OR⁷, j) —NR⁷C(O)NR⁷R⁷, k) —C(S)R⁷, l) —C(S)OR⁷, m) —OC(S)R⁷, n) —C(S)NR⁷R⁷, o) —NR⁷C(S)R⁷, p) —OC(S)NR⁷R⁷, q) —NR⁷C(S)OR⁷, r) —NR⁷C(S)NR⁷R⁷, s) —NR⁷C(NR⁷)NR⁷R⁷, t) —S(O)ₚR⁷, u) —SO₂NR⁷R⁷, and v) R⁷;
R⁴, at each occurrence, independently is selected from the group consisting of:
a) H, b) F, c) Cl, d) Br, e) I, f) ═O, g) ═S, h) ═NR⁵, i) ═NOR⁵, j)═N—NR⁵R⁵, k) —CF₃, l) —OR⁵, m) —CN, n) —NO₂, o) —NR⁵R⁵, p) —C(O)R⁵, q) —C(O)OR⁵, r) —OC(O)R⁵, s) —C(O)NR⁵R⁵, t) —NR⁵C(O)R⁵, u) —OC(O)NR⁵R⁵, v) —NR⁵C(O)OR⁵, w) —NR⁵C(O)NR⁵R⁵, x) —C(S)R⁵, y) —C(S)OR⁵, z) —OC(S)R⁵, aa) —C(S)NR⁵R⁵, bb) —NR⁵C(S)R⁵, cc) —OC(S)NR⁵R⁵, dd) —NR⁵C(S)OR⁵, ee) —NR⁵C(S)NR⁵R⁵, ff) —NR⁵C(NR⁵)NR⁵R⁵, gg) —S(O)ₚR⁵, hh) —SO₂NR⁵R⁵, and ii) R⁵;
R⁵, at each occurrence, independently is selected from the group consisting of:
a) H, b) C₁₋₆ alkyl, c) C₂₋₆ alkenyl, d) C₂₋₆ alkynyl, e) —C(O)—C₁₋₆ alkyl, f) —C(O)—C₂₋₆ alkenyl, g) —C(O)—C₂₋₆ alkynyl, h) —C(O)O—C₁₋₆ alkyl, i) —C(O)O—C₂₋₆ alkenyl, and j) —C(O)O—C₂₋₆ alkynyl,
wherein any of b)-j) optionally is substituted with one or more R⁶ groups;
R⁶, at each occurrence, independently is selected from the group consisting of:
a) F, b) Cl, c) Br, d) I, e) —CF₃, f) —OH, g) —OC₁₋₆ alkyl, h) —SH, i) —SC₁₋₆ alkyl, j) —CN, k) —NO₂, l) —NH₂, m) —NHC₁₋₆ alkyl, n) —N(C₁₋₆ alkyl)₂, o) —C(O)C₁₋₆ alkyl, p) —C(O)OC₁₋₆ alkyl, q) —C(O)NH₂, r) —C(O)NHC₁₋₆ alkyl, s) —C(O)N(C₁₋₆ alkyl)₂, t) —NHC(O)C₁₋₆ alkyl, u) —SO₂NH₂, v) —SO₂NHC₁₋₆ alkyl, w) —SO₂N(C₁₋₆ alkyl)₂, and x) —S(O)ₚC₁₋₆ alkyl;
R⁷, at each occurrence, independently is selected from the group consisting of:
a) H, b) C₁₋₆ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur,
wherein any of b)-p) optionally is substituted with one or more R⁸ groups;
R⁸, at each occurrence, is independently selected from the group consisting of:
a) F, b) Cl, c) Br, d) I, e) ═O, f) ═S, g) ═NR⁹, h) ═NOR⁹, i)═N—NR⁹R⁹, j) —CF₃, k)—OR⁹, l) —CN, m) —NO₂, n) —NR⁹R⁹, o) —C(O)R⁹, p) —C(O)OR⁹, q) —OC(O)R⁹, r) —C(O)NR⁹R⁹, s) —NR⁹C(O)R⁹, t) —OC(O)NR⁹R⁹, u) —NR⁹C(O)OR⁹, v) —NR⁹C(O)NR⁹R⁹, w) —C(S)R⁹, x) —C(S)OR⁹, y) —OC(S)R⁹, z) —C(S)NR⁹R⁹, aa) —NR⁹C(S)R⁹, bb) —OC(S)NR⁹R⁹, cc) —NR⁹C(S)OR⁹, dd) —NR⁹C(S)NR⁹R⁹, ee) —NR⁹C(NR⁹)NR⁹R⁹, ff) —S(O)ₚR⁹, gg) —SO₂NR⁹R⁹, and hh) R⁹;
R⁹, at each occurrence, independently is selected from the group consisting of:
a) H, b) C₁₋₆ alkyl, c) C₂₋₆ alkenyl, d) C₂₋₆ alkynyl, e) C₃₋₁₄ saturated, unsaturated, or aromatic carbocycle, f) 3-14 membered saturated, unsaturated, or aromatic heterocycle comprising one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₃₋₁₄ saturated, unsaturated, or aromatic carbocycle, k) —C(O)-3-14 membered saturated, unsaturated, or aromatic heterocycle comprising one or more heteroatoms selected from the group consisting of nitrogen, oxygen, and sulfur, l) —C(O)O—C₁₋₆ alkyl, m) —C(O)O—C₂₋₆ alkenyl, n) —C(O)O—C₂₋₆ alkynyl, o) —C(O)O—C₃₋₁₄ saturated, unsaturated, or aromatic carbocycle, and p) —C(O)O-3-14 membered saturated, unsaturated, or aromatic heterocycle comprising one or more heteroatoms selected from the group consisting of nitrogen, oxygen, and sulfur,
wherein any of b)-p) optionally is substituted with one or more moieties selected from the group consisting of:
a) F, b) Cl, c) Br, d) I, e) —CF₃, f) —OH, g) —OC₁₋₆ alkyl, h) —SH, i) —SC₁₋₆ alkyl, j) —CN, k) —NO₂, l) —NH₂, m) —NHC₁₋₆ alkyl, n) —N(C₁₋₆ alkyl)₂, o) —C(O)C₁₋₆ alkyl, p) —C(O)OC₁₋₆ alkyl, q) —C(O)NH₂, r)—C(O)NHC₁₋₆ alkyl, s) —C(O)N(C₁₋₆ alkyl)₂, t) —NHC(O)C₁₋₆ alkyl, u) —SO₂NH₂—, v) —SO₂NHC₁₋₆ alkyl, w) —SO₂N(C₁₋₆ alkyl)₂, and x) —S(O)ₚC₁₋₆ alkyl;
m is 0, 1, 2, 3, or 4;
n is 0, 1, 2, 3, or 4; and
p, at each occurrence, independently is 0, 1, or 2.

2. The compound according to claim 1, having the formula: 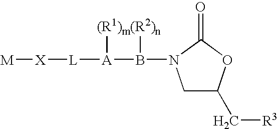
or a pharmaceutically acceptable salt, ester or prodrug thereof,
wherein A, B, L, M, R¹, R², R³, X, m, and n are defined as described in claim 1.

3. The compound according to claim 1, having the formula: 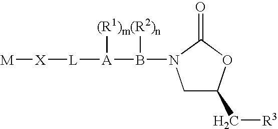
or a pharmaceutically acceptable salt, ester or prodrug thereof,
wherein A, B, L, M, R¹, R², R³, m, and n are defined as described in claim 1.

4. The compound according to claim 1, or a pharmaceutically acceptable salt, ester or prodrug thereof,
wherein
A is selected from the group consisting of phenyl and pyridyl;
B is selected from the group consisting of phenyl and pyridyl;
m is 0, 1, or 2; and
n is 0, 1, or 2.

5. The compound according to claim 1, or a pharmaceutically acceptable salt, ester or prodrug thereof,
wherein A-B is: 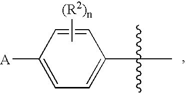
wherein A, R², and n are defined as described in claim 1.

6. The compound according to claim 5 or a pharmaceutically acceptable salt, ester or prodrug thereof, wherein A-B is: 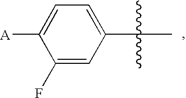
wherein A is defined as described in claim 1.

7. The compound according to claim 5 or a pharmaceutically acceptable salt, ester or prodrug thereof,
wherein A-B is: 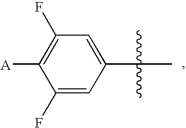
wherein A is defined as described in claim 1.

8. The compound according to claim 1, or a pharmaceutically acceptable salt, ester or prodrug thereof,
wherein A-B is: 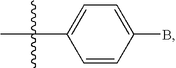
wherein B is defined as described in claim 1.

9. The compound according to claim 1, or a pharmaceutically acceptable salt, ester or prodrug thereof,
wherein A-B is: 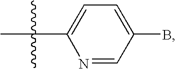
wherein B is defined as described in claim 1.

10. The compound according to claim 1, or a pharmaceutically acceptable salt, ester or prodrug thereof,
wherein R³ is —NHC(O)R⁴.

11. The compound according to claim 10 or a pharmaceutically acceptable salt, ester or prodrug thereof,
wherein R⁴ is —CH₃.

12. The compound according to claim 1, or a pharmaceutically acceptable salt, ester or prodrug thereof,
wherein R³ is:

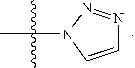

13. The compound according to claim 1, having the formula: 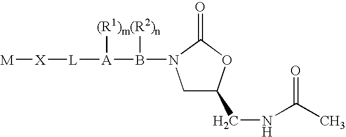
or a pharmaceutically acceptable salt, ester or prodrug thereof,
wherein A, B, L, M, R¹, R², X, m, and n are defined as described in claim 1.

14. The compound according to claim 1, having the formula: 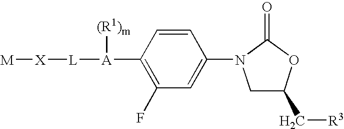
or a pharmaceutically acceptable salt, ester or prodrug thereof,
wherein A, L, M, R¹, R³, X, and m are defined as described in claim 1.

15. The compound according to claim 14, having the formula: 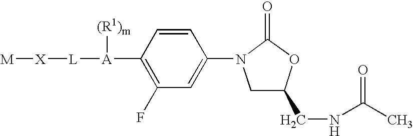
or a pharmaceutically acceptable salt, ester or prodrug thereof,
wherein A, L, M, R¹, X, and m are defined as described in claim 1.

16. The compound according to claim 14, having the formula: 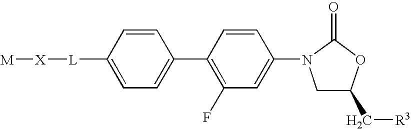
or a pharmaceutically acceptable salt, ester or prodrug thereof,
wherein L, M, R³, and X are defined as described in claim 1.

17. The compound according to claim 16, having the formula: 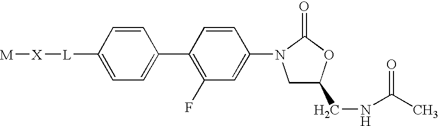
or a pharmaceutically acceptable salt, ester or prodrug thereof,
wherein L, M, and X are defined as described in claim 1.

18. The compound according to claim 14, having the formula: 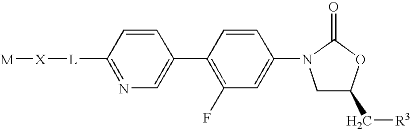
or a pharmaceutically acceptable salt, ester or prodrug thereof,
wherein L, M, R³, and X are defined as described in claim 1.

19. The compound according to claim 18, having the formula: 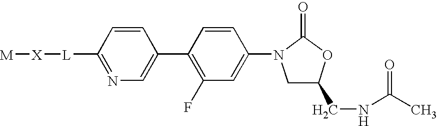
or a pharmaceutically acceptable salt, ester or prodrug thereof,
wherein L, M, and X are defined as described in claim 1.

20. The compound according to claim 1, having the formula: 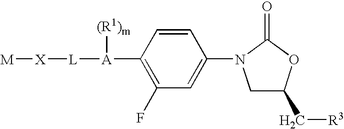
or a pharmaceutically acceptable salt, ester or prodrug thereof,
wherein A, L, M, R¹, R³, X, and m are defined as described in claim 1.

21. The compound according to claim 20, having the formula: 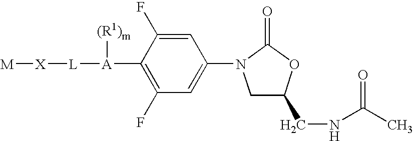
or a pharmaceutically acceptable salt, ester or prodrug thereof,
wherein A, L, M, R¹, X, and m are defined as described in claim 1.

22. The compound according to claim 20, having the formula: 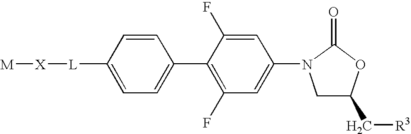
or a pharmaceutically acceptable salt, ester or prodrug thereof,
wherein L, M, R³, and X are defined as described in claim 1.

23. The compound according to claim 22, having the formula: 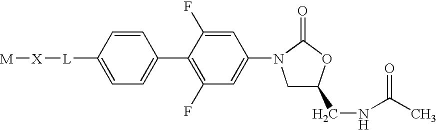
or a pharmaceutically acceptable salt, ester or prodrug thereof,
wherein L, M and X are defined as described in claim 1.

24. The compound according to claim 20, having the formula: 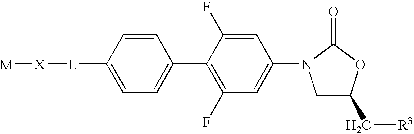
or a pharmaceutically acceptable salt, ester or prodrug thereof,
wherein L, M, R³, and X are defined as described in claim 1.

25. The compound according to claim 24, having the formula: 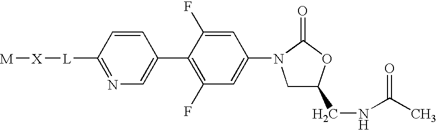
or a pharmaceutically acceptable salt, ester or prodrug thereof,
wherein L, M, and X are defined as described in claim 1.

26. The compound according to claim 1, or a pharmaceutically acceptable salt, ester or prodrug thereof,
wherein L is C₁₋₆ alkyl.

27. The compound according to claim 26 or a pharmaceutically acceptable salt, ester or prodrug thereof,
wherein L is —CH₂—.

28. The compound according to claim 1, or a pharmaceutically acceptable salt, ester or prodrug thereof,
wherein X is —SO₂NH—.

29. The compound according to claim 1, or a pharmaceutically acceptable salt, ester or prodrug thereof,
wherein X is —NHSO₂—.

30. The compound according to claim 1, or a pharmaceutically acceptable salt, ester or prodrug thereof,
wherein X is —SO₂NCH₃—.

31. The compound according to claim 12, or a pharmaceutically acceptable salt, ester or prodrug thereof,
wherein X is —NCH₃SO₂—.

32. The compound according to claim 1, or a pharmaceutically acceptable salt, ester or prodrug thereof,
wherein M is C₁₋₆ alkyl optionally substituted with one or more R⁴ groups.

33. The compound according to claim 32 or a pharmaceutically acceptable salt, ester or prodrug thereof,
wherein M is C₁₋₆ alkyl.

34. The compound according to claim 32 or a pharmaceutically acceptable salt, ester or prodrug thereof,
wherein M is C₁₋₆ alkyl substituted with one or more halogens.

35. A compound having the structure corresponding to any one of the structures listed in Table 1, or a pharmaceutically acceptable salt, ester, or prodrug thereof.

36. A pharmaceutical composition comprising one or more compounds according to claim 1 and a pharmaceutically acceptable carrier.

37. A method of treating a microbial infection in a mammal comprising the step of administering to the mammal an effective amount of one or more compounds according to claim 1.

38. A method of treating a fungal infection in a mammal comprising the step of administering to the mammal an effective amount of one or more compounds according to claim 1.

39. A method of treating a parasitic disease in a mammal comprising the step of administering to the mammal an effective amount of one or more compounds according to claim 1.

40. A method of treating a proliferative disease in a mammal comprising the step of administering to the mammal an effective amount of one or more compounds according to claim 1.

41. A method of treating a viral infection in a mammal comprising the step of administering to the mammal an effective amount of one or more compounds according to claim 1.

42. A method of treating an inflammatory disease in a mammal comprising the step of administering to the mammal an effective amount of one or more compounds according to claim 1.

43. A method of treating a gastrointestinal motility disorder in a mammal comprising the step of administering to the mammal an effective amount of one or more compounds according to claim 1.

44. A method of treating a disorder in a mammal comprising the step of administering to the mammal an effective amount of one or more compounds according to claim 1 thereby to ameliorate a symptom of the disorder, wherein the disorder is selected from the group consisting of:
a skin infection, nosocomial pneumonia, post-viral pneumonia, an abdominal infection, a urinary tract infection, bacteremia, septicemia, endocarditis, an atrio-ventricular shunt infection, a vascular access infection, meningitis, surgical prophylaxis, a peritoneal infection, a bone infection, a joint infection, a methicillin-resistant Staphylococcus aureus infection, a vancomycin-resistant Enterococci infection, a linezolid-resistant organism infection, and tuberculosis.

45. The method according to claim 37, wherein the compound is administered orally, parentally, or topically.

46. A method of synthesizing a compound according to claim 1.

47. A medical device containing one or more compounds according to claim 1.

48. The medical device according to claim 47, wherein the device is a stent.
